(19) United States
(12) Patent Application Publication
(10) Pub. No.: US 2024/0360053 A1
(43) Pub. Date: Oct. 31, 2024

(54) Steam Cracking Processes Having an Elevated Coil Outlet Pressure

(71) Applicant: ExxonMobil Technology and Engineering Company, Baytown, TX (US)

(72) Inventors: Selma S. Lawrence, Houston, TX (US); Hassan Sharifi, The Woodlands, TX (US); Tania M. Almazan, Baton Rouge, LA (US); Priya Rao, Sugarland, TX (US); Mark A. Rooney, Pasadena, TX (US); David Spicer, Houston, TX (US); Stephen R. Phillips, Spring, TX (US)

(21) Appl. No.: 18/623,577

(22) Filed: Apr. 1, 2024

Related U.S. Application Data
(60) Provisional application No. 63/498,290, filed on Apr. 26, 2023.

(51) Int. Cl. C07C 5/327; B01D 3/38; B01J 6/00; C02F 1/04; C02F 101/10; C02F 101/32; C10G 9/36

(52) U.S. Cl. CPC C07C 5/327; B01D 3/38; B01J 6/008; C02F 1/04; C10G 9/36; C02F 2101/101; C02F 2101/32; C10G 2300/1081; C10G 2400/20

(57) ABSTRACT

A process for producing a C2-C4 olefin product from an ethane-containing hydrocarbon feed can be carried out using a steam cracker operated with an elevated coil-outlet pressure, thereby reducing the number of stages of compression required for recovering products from the steam cracker effluent. Coke can be effectively managed in the processes of this disclosure.

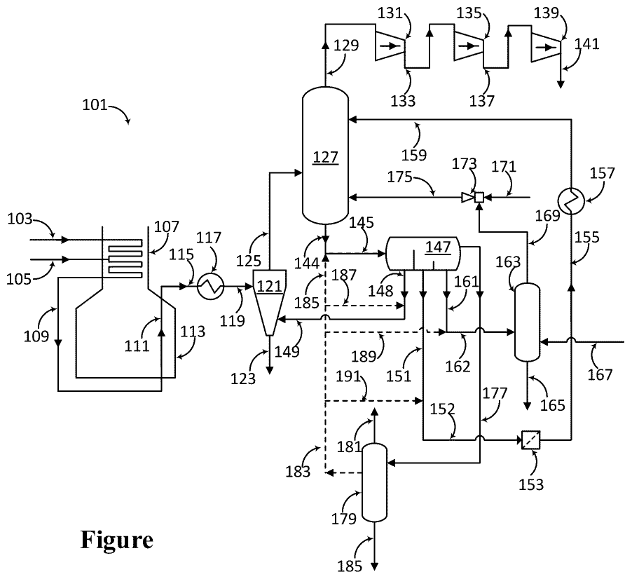

### CROSS-REFERENCE TO RELATED APPLICATION

This application claims priority to and the benefit of U.S. Provisional Application No. 63/498,290 having a filing date of Apr. 26, 2023, the disclosure of which is incorporated herein by reference in its entirety.

### FIELD

This disclosure relates to processes for converting alkane-containing feed to an olefin product using steam cracking. In particular, this disclosure relates to such steam cracking processes having an elevated coil outlet pressure compared to conventional steam cracking processes. The processes are useful, e.g., in converting an ethane-rich hydrocarbon feed into one or more olefin product such as ethylene and/or propylene using steam cracking.

### BACKGROUND

Ethylene and propylene (light olefins) are two of the highest volume petrochemical products manufactured. The polymer products into which they are converted have numerous applications in society ranging from food wrap films that extend produce shelf life to light-weight automotive components that contribute to reduced fuel consumption. The majority of ethylene and propylene are manufactured from hydrocarbon feedstocks by the so-called steam-cracking process in an olefins product plant. In this process the hydrocarbon feed, in the presence of steam, is subjected to pyrolysis at very high temperatures for very short reaction times in radiant coils of a steam cracker, producing a mixed product stream rich in ethylene and propylene, but also containing molecules ranging from hydrogen to fuel-oil. This mixed product stream is then immediately cooled and separated to produce a process gas stream comprising mainly C1-C4 hydrocarbons including ethylene and propylene. The process gas stream is then compressed to a higher pressure, cooled to a very low temperature in a chill train, and separated in distillation columns to recover, among others, an ethylene product stream and a propylene product stream.

The chemical reactions involved in the pyrolysis of the hydrocarbon feed in the steam cracker are highly endothermic and results in a net molar increase of gaseous species. This can be illustrated by the desired reactions for converting ethane to ethylene:

C₂H₆←→CH₂═CH₂+H₂

Therefore, to drive a high conversion of the feed to the desirable olefin product molecules such as ethylene, a lower pressure would be generally preferred, all other conditions held equal. As such, a low pressure in the radiant coils, indicative by a relatively low pressure at the end of the radiant coils called “coil outlet pressure” (“COP”) of no greater than 170 kPa-gauge, has been conventionally used for steam cracking hydrocarbon feeds. This results in a process gas stream with a low pressure in need of compression to the desired high pressure in the recovery section to enable effective separation of the desirable product streams, which can require 4 to 5 stages of compression. Given the very high power rating of the compressors, they are very expensive to procure, install, operate, and maintain. It would be highly desirable to reduce the required compressor stages to conserve capital and operation costs of the olefins production plant.

This disclosure satisfies this and other needs.

### SUMMARY

The present inventors have found that by increasing the COP of the steam cracking process one can reduce the total number of compressor stages required in an olefins production stage. However, the desired steam cracking process conditions including such increased COP can result in the production of elevated levels of tar, which has to be properly managed in the downstream separation processes to prevent fouling. The increased COP additionally poses technical challenges to separation devices handling the steam cracker effluent, such as a water quench tower.

Thus, a first aspect of this disclosure relates to a process comprising one or more of the following: (I) providing the ethane-containing hydrocarbon feed; (II) mixing the ethane-containing hydrocarbon feed with a dilution steam to produce a feed-steam mixture; (III) heating the feed-steam mixture in a convection section of a steam cracking furnace to obtain a heated feed-steam mixture; (IV) cracking the heated feed-steam mixture in a radiant tube located in a radiant section of the steam cracking furnace under pyrolysis conditions to produce a radiant effluent exiting the steam cracking furnace having a coil-outlet pressure (“COP”) of from 200 kPa-gauge to 700 kPa-gauge; (V) cooling the radiant effluent to obtain a cooled effluent; (VI) feeding at least a portion of the cooled effluent into a quench tower; (VII) feeding a quench water stream into the quench tower to contact the cooled effluent in the quench tower; (VIII) obtaining a quench tower overhead vapor stream at a location in the vicinity of the top of the quench tower, and a first quench tower liquid effluent stream at a location in the vicinity of the bottom of the quench tower, from the quench tower; (IX) recovering the C2-C4 olefin product from the quench tower overhead vapor stream by using no more than 3 stages of compression; (X) separating the first quench tower liquid effluent stream optionally mixed with a tar solvent, to obtain a coke-rich stream, a first aqueous stream, an optional second aqueous stream, and an oil stream; (XI) removing at least a portion of the coke contained in the first aqueous stream to obtain a coke-depleted water stream; and (XII) supplying the coke-depleted water stream as at least a portion of the quench water stream in step (VII).

A third aspect of this disclosure relates to a process for producing a C2-C4 olefin product from an ethane-containing hydrocarbon feed, the process comprising one or more of the following: (1) providing the ethane-containing hydrocarbon feed; (2) mixing the ethane-containing hydrocarbon feed with a dilution steam to produce a feed-steam mixture; (3) heating the feed-steam mixture in a convection section of a steam cracking furnace to obtain a heated feed-steam mixture; (4) cracking the heated feed-steam mixture in a radiant tube located in a radiant section of the steam cracking furnace under pyrolysis conditions to produce a radiant effluent exiting the cracking furnace having a coil-outlet pressure (“COP”) of from 200 kPa-gauge to 700 kPa-gauge; (5) cooling the radiant effluent to obtain a cooled effluent; (6) feeding at least a portion of the cooled effluent into a quench tower; (7) feeding a quench water stream into the quench tower to contact the cooled effluent in the quench tower; (8) obtaining a quench tower overhead vapor stream at a location in the vicinity of the top of the quench tower, and a first quench tower liquid effluent stream at a location in the vicinity of the bottom of the quench tower, from the quench tower; (9) processing the quench tower overhead vapor stream to obtain the C2-C4 olefin product by using no more than 3 stages of compression; (10) separating the first quench tower liquid effluent stream optionally mixed with a tar solvent, to obtain a coke-rich stream, a first aqueous stream, a second aqueous stream, and an oil stream; and (11) stripping the second aqueous stream in a stripping column at a column bottom temperature of no greater than 280° F. and an stripping column overhead pressure of no greater than 250 kPa-gauge to obtain a stripping column overhead vapor stream and a stripping column bottoms process water stream; and (12) forming a stripping column recycle stream having a higher pressure than the stripping column overhead vapor stream from the stripping column overhead vapor stream; and (13) feeding the stripping column recycle stream into the quench tower.

A fourth aspect of this disclosure relates to a process for producing a C2-C4 olefin product from an ethane-containing hydrocarbon feed, the process comprising: (a) providing the ethane-containing hydrocarbon feed; (b) mixing the ethane-containing hydrocarbon feed with a dilution steam to produce a feed-steam mixture; (c) heating the feed-steam mixture in a convection section of a steam cracking furnace to obtain a heated feed-steam mixture; (d) cracking the heated feed-steam mixture in a radiant tube located in a radiant section of the steam cracking furnace under pyrolysis conditions to produce a radiant effluent exiting the cracking furnace having a coil-outlet pressure (“COP”) of from 200 kPa-gauge to 700 kPa-gauge; (c) cooling the radiant effluent to obtain a cooled effluent; (f) feeding at least a portion of the cooled effluent into a quench tower by: (f.1) providing a separation device comprising a feed stream inlet, a vapor outlet, and a coke particle deposition section; (f.2) feeding the cooled effluent into the feed stream inlet; (f.3) obtaining a coke-depleted vapor stream from the vapor outlet as the coke-abated cooled effluent stream; and (f.4) obtaining deposited coke from the coke deposition section; (g) feeding a quench water stream into the quench tower to contact the cooled effluent in the quench tower; (h) obtaining a quench tower overhead vapor stream at a location in the vicinity of the top of the quench tower, and a first quench tower liquid effluent stream at a location in the vicinity of the bottom of the quench tower, from the quench tower; (i) processing the quench tower overhead vapor stream to obtain the C2-C4 olefin product by using no more than 3 stages of compression; and (j) separating the first quench tower liquid effluent stream optionally mixed with a tar solvent, to obtain a coke-rich stream, a first aqueous stream, a second aqueous stream, and an oil stream.

A fifth aspect of this disclosure relates to a process for producing a C2-C4 olefin product from an ethane-containing hydrocarbon feed, the process comprising one or more of the following: (A) providing the ethane-containing hydrocarbon feed; (B) mixing the ethane-containing hydrocarbon feed with a dilution steam to produce a feed-steam mixture; (C) heating the feed-steam mixture in a convection section of a steam cracking furnace to obtain a heated feed-steam mixture; (D) cracking the heated feed-steam mixture in a radiant tube located in a radiant section of the steam cracking furnace under pyrolysis conditions to product a radiant effluent exiting the cracking furnace having a coil-outlet pressure (“COP”) of from 200 kPa-gauge to 700 kPa-gauge; (E) cooling the radiant effluent to obtain a cooled effluent; (F) feeding at least a portion of the cooled effluent into a quench tower; (G) feeding a quench water stream into the quench tower to contact the cooled effluent in the quench tower; (H) obtaining a quench tower overhead vapor stream at a location in the vicinity of the top of the quench tower, and a first quench tower liquid effluent stream at a location in the vicinity of the bottom of the quench tower, from the quench tower; (I) processing the quench tower overhead vapor stream to obtain the C2-C4 olefin product by using no more than 3 stages of compression; (J) separating the first quench tower liquid effluent stream optionally mixed with a tar solvent, to obtain a coke-rich stream, a first aqueous stream, a second aqueous stream, and an oil stream; and (K) separating from the oil stream a C6-C7 aromatics-rich stream; and (L) mixing at least a portion of the C6-C7 aromatics-rich stream with at least one of the following: (i) the first quench tower liquid effluent stream as at least a portion of the tar solvent in step (J); (ii) the coke-rich stream after step (J); (iii) the aqueous stream after step (J).

A sixth aspect of this disclosure relates to a process for producing a C2-C4 olefin product from an ethane-containing hydrocarbon feed, the process comprising one of more of the following: (i) providing the ethane-containing hydrocarbon feed; (ii) mixing the ethane-containing hydrocarbon feed with a dilution steam to produce a feed-steam mixture; (iii) heating the feed-steam mixture in a convection section of a steam cracking furnace to obtain a heated feed-steam mixture; (iv) cracking the heated feed-steam mixture in a radiant tube located in a radiant section of the steam cracking furnace under pyrolysis conditions to product a radiant effluent exiting the cracking furnace having a coil-outlet pressure (“COP”) of from 200 kPa-gauge to 700 kPa-gauge; (v) cooling the radiant effluent to obtain a cooled effluent; (vi) feeding at least a portion of the cooled effluent into a quench tower by: (vi.1) providing a separation device comprising a feed stream inlet, a vapor outlet, and a coke particle deposition section; (vi.2) feeding the cooled effluent into the feed stream inlet; (vi.3) obtaining a coke-depleted vapor stream from the vapor outlet as the coke-abated cooled effluent stream; and (vi.4) obtaining deposited coke from the coke deposition section; (vii) feeding a quench water stream into the quench tower to contact the cooled effluent in the quench tower; (viii) obtaining a quench tower overhead vapor stream and a quench tower lower liquid stream from the quench tower; (ix) processing the quench tower overhead vapor stream to obtain the C2-C4 olefin product by using no more than 3 stages of compression; (x) separating the first quench tower liquid effluent stream optionally mixed with a tar solvent, to obtain a coke-rich stream, a first aqueous stream, a second aqueous stream, and an oil stream; (xi) separating from the oil stream a C6-C7 aromatics-rich stream; and (xii) mixing at least a portion of the C6-C7 aromatics-rich stream with at least one of the following: (a) the first quench tower liquid effluent stream as at least a portion of the tar solvent in step (x); (b) the coke-rich stream after step (x); (c) the aqueous stream after step (x); (xiii) stripping the second aqueous stream in a stripping column at a column bottom temperature of no greater than 280° F. and an stripping column overhead pressure of no greater than 250 kPa-gauge to obtain a stripping column overhead vapor stream and a stripping column bottoms process water stream; (xiv) forming a stripping column recycle stream having a higher pressure than the stripping column overhead vapor stream from the stripping column overhead vapor stream; and (xv) feeding the stripping column recycle stream into the quench tower.

### BRIEF DESCRIPTION OF THE DRAWING

The FIGURE schematically illustrates various embodiments of the various aspects of the processes of this disclosure.

### DETAILED DESCRIPTION

Various specific embodiments, versions and examples of the invention will now be described, including preferred embodiments and definitions that are adopted herein for purposes of understanding the claimed invention. While the following detailed description gives specific preferred embodiments, those skilled in the art will appreciate that these embodiments are exemplary only, and that the invention may be practiced in other ways. For purposes of determining infringement, the scope of the invention will refer to any one or more of the appended claims, including their equivalents, and elements or limitations that are equivalent to those that are recited. Any reference to the “invention” may refer to one or more, but not necessarily all, of the inventions defined by the claims.

In this disclosure, a process is described as comprising at least one “step.” It should be understood that each step is an action or operation that may be carried out once or multiple times in the process, in a continuous or discontinuous fashion. Unless specified to the contrary or the context clearly indicates otherwise, multiple steps in a process may be conducted sequentially in the order as they are listed, with or without overlapping with one or more other steps, or in any other order, as the case may be. In addition, one or more or even all steps may be conducted simultaneously with regard to the same or different batch of material. For example, in a continuous process, while a first step in a process is being conducted with respect to a raw material just fed into the beginning of the process, a second step may be carried out simultaneously with respect to an intermediate material resulting from treating the raw materials fed into the process at an earlier time in the first step. Preferably, the steps are conducted in the order described.

Unless otherwise indicated, all numbers indicating quantities in this disclosure are to be understood as being modified by the term “about” in all instances. It should also be understood that the precise numerical values used in the specification and claims constitute specific embodiments. Efforts have been made to ensure the accuracy of the data in the examples. However, it should be understood that any measured data inherently contains a certain level of error due to the limitation of the technique and/or equipment used for acquiring the measurement.

Certain embodiments and features are described herein using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated.

The indefinite article “a” or “an”, as used herein, means “at least one” unless specified to the contrary or the context clearly indicates otherwise. Thus, embodiments using “an olefin product” include embodiments where one, two or more olefin products may be produced, unless specified to the contrary or the context clearly indicates that only one olefin product is produced.

The term “hydrocarbon” means (i) any compound consisting of hydrogen and carbon atoms or (ii) any mixture of two or more such compounds in (i). The term “Cn hydrocarbon,” where n is a positive integer, means (i) any hydrocarbon compound comprising carbon atom(s) in its molecule at the total number of n, or (ii) any mixture of two or more such hydrocarbon compounds in (i). Thus, a C2 hydrocarbon can be ethane, ethylene, acetylene, or mixtures of at least two of these compounds at any proportion. A “Cm to Cn hydrocarbon” or “Cm-Cn hydrocarbon,” where m and n are positive integers and m<n, means any of Cm, Cm+1, Cm+2, . . . , Cn−1, Cn hydrocarbons, or any mixtures of two or more thereof. Thus, a “C2 to C3 hydrocarbon” or “C2-C3 hydrocarbon” can be any of ethane, ethylene, acetylene, propane, propene, propyne, propadiene, cyclopropane, and any mixtures of two or more thereof at any proportion between and among the components. A “saturated C2-C3 hydrocarbon” can be ethane, propane, cyclopropane, or any mixture thereof of two or more thereof at any proportion. A “Cn+ hydrocarbon” means (i) any hydrocarbon compound comprising carbon atom(s) in its molecule at the total number of at least n, or (ii) any mixture of two or more such hydrocarbon compounds in (i). A “Cn− hydrocarbon” means (i) any hydrocarbon compound comprising carbon atoms in its molecule at the total number of at most n, or (ii) any mixture of two or more such hydrocarbon compounds in (i). A “Cm hydrocarbon stream” means a hydrocarbon stream consisting essentially of Cm hydrocarbon(s). A “Cm-Cn hydrocarbon stream” means a hydrocarbon stream consisting essentially of Cm-Cn hydrocarbon(s).

“Consisting essentially of” means comprising ≥60 mol %, preferably ≥75 mol %, preferably ≥80 mol %, preferably ≥90 mol %, preferably ≥95 mol %; preferably 98 mol %, of a given material or compound, in a stream or mixture, based on the total moles of molecules in the stream or mixture.

In this disclosure, any mention of coke, tar, coke/tar, or tar/coke should be understood to mean coke, tar, or a mixture thereof, as the case may be.

In this disclosure, “in the vicinity” of the bottom of a column means at the bottom of the column, or at a location above the bottom of the column, but within the lowest ⅓ of the column, preferably within the lowest ¼ of the column. “In the vicinity” of the top of a column means at the top of the column, or at a location below the top of the bottom, but within the highest ⅓ of the column, preferably within the highest ¼ of the column.

For the purposes of this disclosure, the nomenclature of elements is pursuant to the version of the Periodic Table of Elements (under the new notation) as provided in Hawley's Condensed Chemical Dictionary, 16^{th} Ed., John Wiley & Sons, Inc., (2016), Appendix V.

The hydrocarbon feed used in the processes of this disclosure can be any hydrocarbon feed suitable for steam cracking. It can consist essentially of hydrocarbons. It can further comprise minor components such as hydrocarbon derivatives. It can be a gas feed consisting essentially of C2-C4 hydrocarbons, or a liquid feed such as a naphtha feed, a gas oil feed, or a crude feed. Preferably however, the hydrocarbon feed used in the processes of this disclosure is a gas feed. Preferably, the hydrocarbon feed consists essentially of C2-C4 hydrocarbons. The hydrocarbon feed can comprise, consist essentially of, or consist of a neat feed provided from a feed source external to the olefins production plant. The hydrocarbon feed can comprise, consist essentially of, or consist of a recycle stream produced from the recovery section of an olefins production plant. The hydrocarbon feed can comprise a mixture of a neat feed provided a feed external to the olefins production plant and a recycle stream produced from the recovery section of the olefins production plant at any ration between these two. Preferably, the hydrocarbon feed used in the processes of this disclosure comprises ethane at a molar concentration of ≥50%, ≥60%, ≥70%, ≥80%, ≥85%, ≥90%, ≥95%, ≥98 %, or even >99%, and up to 100%, based on the total moles of species in the hydrocarbon feed. The processes of this disclosure are particularly advantageous for such as hydrocarbon feed rich in ethane. In certain embodiments, the hydrocarbon feed used in the processes of this disclosure comprises propane at a molar concentration of ≥50%, ≥60%, ≥70%, ≥80%, ≥85%, ≥90%, ≥95%, ≥98%, or even ≥99%, and up to 100%, based on the total moles of species in the hydrocarbon feed. In certain other embodiments, the hydrocarbon feed used in the processes of this disclosure comprises propane at a molar concentration of ≥50%, ≥60%, ≥70%, ≥80%, ≥85%, ≥90%, ≥95%, ≥98%, or even ≥99%, and up to 100%, based on the total moles of species in the hydrocarbon feed. In yet other embodiments, the hydrocarbon feed used in the processes of this disclosure comprises ethane and propane, in combination, at a molar concentration of ≥50%, ≥60%, ≥70%, ≥80%, ≥85%, ≥90%, ≥95%, ≥98%, or even ≥99%, and up to 100%, based on the total moles of species in the hydrocarbon feed. In certain embodiments, the hydrocarbon feed is a neat ethane feed provided from a source external to the olefins production plant. In other embodiments, the hydrocarbon feed is a mixture comprising both a neat ethane feed provided from a source external to the olefins production plant and a recycle ethane stream produced from the recovery section of the olefins production plant, at any ratio between these two.

The hydrocarbon feed can be first fed into a heat exchanger tube, where it is heated. Preferably the heat exchanger tube is located in the convection section of a steam cracking furnace. The heated hydrocarbon feed can then be mixed with a dilution steam to produce a feed-stream mixture. The weight ratio of the dilution steam to the hydrocarbon feed can range from, e.g., r1 to r2, where r1 and r2 can be, independently, e.g., 0.1, 0.2, 0.3, 0.4, 0.5, 0.6, 0.7, 0.8, 0.9, 1, as long as r1<r2. The feed-steam mixture can then be further heated in a heat exchanger, preferably one located in the convection section of a steam cracking furnace, to obtain a heated feed-steam mixture. The heated feed-steam mixture can have a temperature ranging from T1 to T2° C., where T1 and T2 can be, independently, 690, 700, 710, 720, 730, 740, 750, 760, 770, 771, 775, 780, as long as T1<T2. The thus heated feed-steam mixture can be sent to one or more radiant tubes located in the radiant section of a steam cracking furnace and cracked. A pipe located outside of the steam cracking furnace, called cross-over section tube, may be used for supplying the heated feed-steam mixture to the radiant tube(s). At a relatively high temperature in the cross-over section, some of the hydrocarbon molecules in the heated feed-steam mixture may undergo pyrolysis reactions (i.e., cracking reactions) to a limited extent. Such limited cracking in the cross-over section can be advantageous in certain embodiments, as it can reduce the severity required in the radiant tube with limited amount of tar formation in the cross-over section and reduced amount of tar formation in the radiant tube.

The radiant tubes are typically heated to a high temperature in the radiant section of the steam cracking furnace, by, e.g., the thermal energy released by combusting a fuel such as natural gas, hydrogen, a mixture thereof, and the like. The heated feed-mixture, once entering the radiant tube(s), is quickly heated to an elevated pyrolysis temperature, where at least a majority of the pyrolysis reactions occur for a short residence time ranging from t1 to t2 second, where t1 and t2 can be, independently, e.g., 0.01, 0.02, 0.03, 0.04, 0.05, 0.06, 0,.07, 0.08, 0.09, 0.1, 0.2, 0.3, 0.4, 0.5, 0.6, 0.7, 0.8, 0.9, 1, as long as t1<t2. Preferably t1=0.1, and t2=0.3. The pyrolysis reactions produce, in addition to hydrogen, the desired olefins such as ethylene, propylene, and the butenes, saturated and unsaturated naphtha range hydrocarbons, saturated and unsaturated gas oil range hydrocarbons, and tar/coke. At higher cracking severity, more tar/coke can be produced.

As discussed above, in the processes of this disclosure, a high pyrolysis pressure in the radiant tube(s) is utilized in order to achieve a high COP that enables reduction of number of stages of compressors required in the recovery section of the olefins production plant. Because the pyrolysis reactions result in a next increase of gases species in the reaction mixture, a higher pyrolysis pressure—while all other conditions held equal—reduces the conversion of alkanes in the hydrocarbon feed to the desirable olefins molecules. Thus, in certain embodiments, in order to increase conversion of the alkanes in the hydrocarbon feed to the valuable olefins molecules, a higher pyrolysis temperature (i.e., a higher cracking severity) and/or a longer residence time in the radiant tube(s) may be utilized, which can result in the production of more tar/coke. The increase tar/coke production in the radiant tube(s) can cause fouling in the radiant tube(s) and downstream processes and equipment. To prevent fouling in the radiant tube, additional coke mitigation can be provided by using inner surfaces within the furnace coils that are resistant to coke formation and/or are resistant to carburization. Such materials can include, but are not limited to, nickel-chromium alloys that optionally further include an alumina barrier layer. Examples of such materials are described in U.S. Pat. Nos. 8,431,230 and 10,041,152, which are incorporated by reference herein in their entireties. In certain advantageous embodiments, the radiant tube(s) can comprise:
a cast body of a heat-resistant alloy consisting essentially of, in mass percent, 0.05 to 0.7% of C, more than 0% and no more than 2.5% of Si, more than 0% and no more than 3.0% of Mn, 15 to 50% of Cr, 20 to 70% of Ni, 2 to 4% of Al, 0.005 to 0.4% of rare-earth elements, and at least one member selected from the group consisting of 0.5 to 10% of W and 0.1 to 5% of Mo, the balance being Fe and inevitable impurities; and
a barrier layer formed on a surface of the cast body to be brought into contact with the high temperature atmosphere;
wherein:
the barrier layer preferably comprises, consists essentially or, or consists of an Al₂O₃ layer having a thickness of 0.5 μm or more;
preferably at least 80 area % of an outermost surface of the cast body is Al₂O₃;
preferably the cast product has Cr-based particles dispersed at an interface between the Al₂O₃ layer and the cast body at a higher Cr concentration than a Cr concentration of a matrix of the alloy; and
preferably the surface of the cast body before forming Al₂O₃ layer is of a roughness (Ra) of 0.05 to 2.5.

Description such advantages cast body and radiant tubes can be found, e.g., U.S. Pat. No. 8,431,230, the relevant disclosure of which are incorporated herein by reference in its entirety. At the end of the radiant tube(s) (called “coil outlet”), a pyrolysis effluent exits the steam cracking furnace. The pyrolysis effluent comprises hydrogen, C1-C4 hydrocarbons such as methane, ethane, ethylene, propane, propylene, butenes, and butadienes, steam cracker naphthas, steam cracker gas oil, and steam cracker tar/coke. In various embodiments of the processes of this disclosure, the pyrolysis effluent at the coil outlet has a coil outlet pressure (“COP”) ranging from COP1 to COP2 kPa-gauge, where COP1 and COP2 can be, independently, e.g., 200, 210, 220, 230, 240, 250, 260, 270, 280, 290, 300, 320, 340, 350, 360, 380, 400, 420, 430, 440, 450, 460, 470, 480, 490, 500, 510, 520, 540, 550, 560, 580, 600, 620, 640, 650, 660, 680, 700, as long as COP1<COP2. In advantageous embodiments COP1=270, and COP2=520. In other advantageous embodiments, COP1=300, and COP2=400.

To minimize undesirable reactions between and among the reactive species present in the pyrolysis effluent at elevated temperature, the pyrolysis effluent is typically immediately cooled by direct cooling (e.g., by mixing with a quench oil having a much lower temperature) and/or indirectly cooling (e.g., by using a heat exchanger such as a transfer line heat exchanger “(TLE”)), to produce a cooled pyrolysis effluent. The cooled pyrolysis effluent, with optional additional direct and/or indirect cooling, can then be separated to obtain: a process gas stream rich in hydrogen and C1-C4 hydrocarbons; one or more steam cracker naphtha stream; one or more steam cracker gas oil stream; and one or more steam cracker tar/coke stream.

In certain embodiments of the processes of this disclosure, particularly those utilizing a hydrocarbon feed comprising ≥50 mol % of ethane, the cooled pyrolysis effluent may be processed to remove a portion of the tar/coke contained in the cooled effluent to obtain a coke-abated cooled effluent stream to reduce/prevent fouling of downstream process equipment caused by the tar/coke. To that end, a coke-separation device comprising a feed stream inlet, a vapor outlet, and a tar deposition section may be used. The cooled pyrolysis effluent can be fed into the feed stream inlet, a coke-depleted vapor stream is obtained from the vapor outlet as the coke-abated cooled effluent stream, and a portion of the coke/tar present in the cooled pyrolysis effluent can be separated and obtained from the coke/tar deposition section. An example of a useful coke-separation device can be a coke catchleg, the structure and operation of which can be found in, e.g., WO2022/150218, the relevant of which is incorporated herein by reference in its entirety. Another example of a useful coke-separation device can be a centrifuge, the structure and operation of which can be found in, e.g., WO2020/168062, the relevant portion of which is incorporated herein by reference in its entirety. The coke-depleted vapor stream may nonetheless comprise coke/tar at a significant amount, which can be advantageously mitigated by the various processes steps as described and illustrated below in this application to further prevent or reducing fouling of downstream process equipment.

In certain embodiments, the cooled effluent and/or the coke-abated cooled effluent can then be fed into a quench tower, preferably at a lower location of the quench tower, where it contacts, preferably in a counter-current manner, a quench water fed into the quench tower, preferably at an upper location of the quench tower above the cooled effluent inlet, and further quenched and washed. From the top or in the vicinity of the top of the quench tower, a quench tower overhead vapor stream is obtained, comprising steam, hydrogen, C1-C4 hydrocarbons including but not limited to desirable molecules such as ethylene, propylene, and butenes, various minor amounts of CO, CO₂, H₂S, and C5+ hydrocarbons. The quench tower overhead vapor stream can desirably have a pressure approximate that of COP, i.e., ranging from COP1 to COP2 kPa-gauge, where COP1 and COP2 can be, independently, e.g., 200, 210, 220, 230, 240, 250, 260, 270, 280, 290, 300, 320, 340, 350, 360, 380, 400, 420, 430, 440, 450, 460, 470, 480, 490, 500, 510, 520, 540, 550, 560, 580, 600, 620, 640, 650, 660, 680, 700, as long as COP1<COP2. In advantageous embodiments COP1=270, and COP2=520. In other advantageous embodiments, COP1=300, and COP2=400.

The quench tower overhead vapor stream can be subjected to various treatment and separation to produce the desirable products such as hydrogen, ethylene, propylene, butenes, butadiene, and the like, and byproducts such as tailgas, ethane, water, CO₂, H₂S, and C5+, and the like. Such treatment and separation can include no more than four stages of compression using compressors, preferably no more than three stages of compression, and one or more of the following before and/or after each stage of compression: condensing, settling, cooling, heating, distilling, drying, sour-gas removing, selective hydrogenation (e.g., to abate alkynes and dienes). As discussed earlier, energy-efficient distillation separation of such products such as hydrogen, tailgas, ethylene, ethane, and propylene, which have very low normal boiling points, requires compressing gas mixtures (e.g., various fractions of the quench tower overhead vapor stream) to various elevated pressures in addition to cooling them to a low temperature. The high pressure of the quench tower overhead vapor stream enables compression to a desirable pressure using no more than 3 stages of compression. In certain desirable embodiments, three sequential stages of compression are used, consisting of a first stage, a second stage downstream of the first stage, and a third stage downstream of the second stage. In various embodiments, the first stage compressor can have a first stage outlet pressure ranging from P(s1)1 kPa-gauge to P(s1)2 kPa-gauge, where P(s1)1 and P(s1)2 can be, independently, e.g., 310, 320, 340, 350, 360, 380, 400, 450, 500, 550, 600, 650, 700, 750, 850, 860, 880, 900, 920, 940, 940, 960, as long as P(s1)<P(s2). In various embodiments, the second stage compressor can have a second stage outlet pressure higher than the first stage outlet pressure, ranging from P(s2)1 kPa-gauge to P(s2)2 kPa-gauge, where P(s2)1 and P(s2)2 can be, independently, e.g., 510, 520, 540, 550, 560, 580, 600, 650, 700, 800, 900, 1000, 1100, 1200, 1300, 1400, 1500, 1600, 1700, 1750, 1760, 1780, 1800, 1810, 1820, 1830, as long as P(s1)2<P(s2)1<P(s2)2. In various embodiments, the third stage compressor can have a third stage outlet pressure higher than the second stage outlet pressure, ranging from P(s3)1 kPa-gauge to P(s3)2 kPa-gauge, where P(s3)1 and P(s3)2 can be, independently, e.g., 930, 940, 950, 960, 980, 1000, 1200, 1400, 1500, 1600, 1800, 2000, 2400, 2500, 2800, 3000, 3200, 3500, 3520, 3540, 3550, 3560, 3580, 3600, 3610, 3620, 3630, 3700, 3800, 3900, 4000, 4100, 4200, as long as P(s2)2<P(s3)1<P(s3)2.

A first quench tower liquid stream can be drawn from a location in the vicinity of the bottom of the quench tower. In certain embodiments, the first quench tower liquid stream is drawn from the bottom of quench tower and comprises coke/tar at a relatively high concentration based on the total weight of the first quench tower liquid stream. In other embodiments, the first quench tower liquid stream is drawn not from the bottom of the quench tower, but from a location above the bottom of the quench tower, e.g., as a side draw from the quench tower. In such embodiments, it may be advantageous to draw a second quench tower liquid stream from a location below the first quench tower liquid stream, e.g., from the bottom of the quench tower. The lower, second quench tower liquid stream may comprise coke at a higher concentration, based on the total weight of the second quench tower liquid stream, than the first quench tower liquid stream, based on the total weight of the first quench tower liquid stream.

The first quench tower liquid stream comprises steam cracker naphtha, steam cracker gas oil, steam cracker tar, coke, and water. The first quench tower liquid stream can be desirably separated in a separation device to obtain a coke-rich stream, a first aqueous stream, an optional second aqueous stream, and an oil stream. A particularly advantageous separation device for that operation is a settling drum having multiple separation zones optionally separated by a baffle. Thus, advantageously, the first quench tower liquid stream is fed, via an inlet, into a settling drum, in which the heavies components such as coke settle to the bottom of the drum (e.g., one or more boots installed at the bottom) in a first separation zone close to the inlet, and withdrawn as the coke-rich stream. A substantial amount of the water contained in the first quench tower liquid can settle to the bottom of a second separation zone and withdrawn as the first aqueous stream. The second separation zone can be downstream of the first separation zone. The second separation zone can be separated from the first separation zone by a baffle that may be perforated to facilitate the effective settling and separation of coke in the first separation zone. The first aqueous stream can comprise, in addition to water, coke/tar, and minor quantities of steam cracker naphtha and steam cracker gas oil. In certain embodiments, a second aqueous stream may be separated from a third separation zone in the settling drum downstream of the second separation zone. The third separation zone may be separated by an optionally perforated baffle from the second separation zone. The second aqueous stream can comprise, in additional to water, coke/tar (preferably at a lower concentration thereof than in the first aqueous stream), steam cracker naphtha, and steam cracker gas oil. From a fourth separation zone downstream of the third separation zone in the quench separation drum, an oil stream can be obtained. The fourth separation may be separated from the third separation zone by a baffle. In certain other embodiments, no such second aqueous stream is obtained from the third separation zone, and instead only the oil stream is obtained from the third separation zone.

The presence of coke/tar in the first quench tower liquid stream and the optional second quench tower liquid stream can cause fouling in (i) the pipeline containing them, such as the pipeline feeding the first quench tower liquid stream into the quench separation drum and/or (ii) the downstream equipment such as the quench separation drum, especially where the first quench tower liquid stream comprises coke/tar at a high concentration, e.g., where the first quench tower liquid stream is drawn from the bottom of the quench tower. The presence of coke/tar in the first aqueous stream, the optional second aqueous stream, and the oil stream, can also cause fouling in the pipeline for these streams and downstream equipment. To reduce and/or prevent fouling, in certain embodiments, it is desirable to feed a stream of tar solvent into at least one of the following: (i) the pipeline feeding the first aqueous stream into the quench separation drum, (ii) the pipeline containing the second quench tower liquid stream; (ii) the quench separation drum, (iii) the pipeline containing the coke-rich stream; (iv) the pipeline containing the first aqueous stream; (v) the pipeline containing the second aqueous stream, continuously or intermittently, to solvate and/or remove at least a portion of the coke that may be present in the first quench tower liquid stream, the second quench tower liquid stream, or on the inner surface of any of the pipeline or the quench separation drum. The tar solvent can comprise a hydrocarbon or a mixture of a hydrocarbons capable of solvating at least a portion of the coke/tar present in the various streams or on the various surfaces. Advantageously, the tar solvent comprises an aromatic hydrocarbon such as C6-C7 aromatics. A non-limiting example of tar solvent is a naphtha stream, such as a steam cracker naphtha stream, produced in a hydrocarbon processing plant such as a steam cracking-based olefins production plant. In another example, a stream rich in C6-C7 aromatics obtainable from the oil stream, as disclosed in more detail later in this application, may be advantageously used as at least a portion of the tar solvent.

As described above, the first aqueous stream may comprise a substantial quantity of coke/tar. It is highly desirable to remove at least a portion of the coke/tar contained therein to reduce/prevent fouling of downstream equipment receiving or processing the first aqueous stream. In certain embodiments, it is highly advantageous to recycle at least a portion of the water contained in the first aqueous stream into the quench tower as at least a portion of the quench water stream. In such embodiments, it is highly desirable to remove at least a portion of the coke/tar in the first aqueous stream to obtain a coke-depleted water stream, and then supply at least a portion of the coke-depleted water stream to the quench tower as at least a portion of the quench water stream, in order to prevent accumulation of coke in the quench tower and potential fouling of the quench tower.

Various means may be used to remove at least a portion coke from the first aqueous stream. In certain embodiments, a filter may be used.

The optional second aqueous stream, if obtained, may be partially conducted away as a wastewater stream. In certain embodiments, it is desirable that the water contained in the second aqueous stream is at least partly reused, e.g., for generating steam, for use in other process steps, such as for mixing with a steam cracking hydrocarbon feed to form the feed-steam mixture as discussed earlier. The second aqueous stream can comprise, in additional to water, a quantity of hydrocarbons such as steam cracker naphtha and various amount of coke/tar, and a quantity of sour gases (CO₂, H₂S). Before supplying the second aqueous stream to a steam generator such as dilution steam generator, it is highly desirable to strip it to remove or reduce the hydrocarbon and/or sour gas contained therein. To that end, the second aqueous steam may be fed into a stripping column, where it contacts with stripping steam to produce a stripping column overhead vapor stream and a stripping column bottoms process water stream. The stripping steam can be preferably supplied from a steam source. Alternatively, the stripping steam can be partially or entirely generated in situ by, e.g., a reboiler equipped with the stripping column. Preferably, to prevent fouling in the stripping column that may be caused by foulant present therein, e.g., styrene and derivatives thereof, it is highly desirable that the stripping column is operated to have a column bottom temperature no greater than 280° F. (138° C.). To achieve such relatively low bottoms temperature, it is highly desirable that the stripping column is operated under a stripping column overhead pressure of no greater than 250 kPa-gauge, e.g., from 25, 26, 27, 30, 35, 40, 45, 50 kPa-gauge, to 60, 70, 80, 90, 100 kPa-gauge, to 110, 120, 130, 140, 150 kPa-gauge, to 160, 170, 180, 190, 200 kPa-gauge, to 210, 220, 230, 240, 250 kPa-gauge, preferably from 27 to 96 kPa-gauge.

It is highly desirable to feed the stripping column overhead stream into the quench tower. In certain embodiments, the quench tower may have an internal pressure higher than that the stripping column overhead stream. In such case, the stripping column overhead stream can be converted to a high-pressure stripping column recycle stream, by, e.g., compressing using a compressor, and/or by joining with a higher-pressure stream, which is then fed into the quench tower. In a particularly advantageously embodiment, the stripping column overhead stream can be fed into an ejector, along with a higher-pressure stream (e.g., a steam stream, a hydrocarbon stream, and mixtures thereof) as a movant, to produce the stripping column recycle stream suitable for feeding into the quench tower.

The stripping column bottoms process water stream can then be fed into a steam generator to generate steam suitable for use as at least a portion of the dilution steam to mix with the hydrocarbon feed to a steam cracker.

The oil stream obtainable from the quench separation drum as described above comprises steam cracker naphtha, steam cracker gas oil, and steam cracker tar. Thus, one or more steam cracker naphtha streams, one or more steam cracker gas oil streams, and one or more steam cracker tar streams can be produced from the oil stream by using various separation means such as distillation, extraction, and the like. Upon optional additional treatment, such as hydrotreating, these streams can be used as gasoline blend stocks, diesel blend stocks, and fuel oil blending stocks. In one particularly advantageous embodiment, a hydrocarbon stream rich in C6-C7 aromatic hydrocarbons can be separated from the oil stream. The C6-C7 aromatics-rich stream can be advantageously produced with the quality and quantity particularly suitable as a tar solvent for solvating coke/tar in various streams as described above. This in-situ generation of tar solvent can eliminate the need for sourcing such tar solvent from outside of the steam-cracker-based olefins production plant, and is therefore particularly cost-effective.

Various preferred embodiments of the processes of this disclosure are described and illustrated below by reference to the FIGURE. It should be noted that the FIGURE is for illustration purpose and only and should be interpreted to limit the inventions as claimed in this application.

### THE FIGURE

The FIGURE illustrates various embodiments 101 of the processes of this disclosure. It should be understood that this and other drawings are just schematic depiction for the purpose of illustrating the operation principles and process flows. Many process equipment, such as valves, pumps, meters, heat exchangers, reboilers, reflux drums, and the like, are omitted. One having ordinary skill in the art may choose to add those additional equipment without departing from the scope of this disclosure.

As shown in the FIGURE, an ethane-containing hydrocarbon feed 103 is fed into heat exchange tubes located in a convection section 107 of a steam cracking furnace, where it is heated, and then combined with a dilution steam stream 105 to form a feed-steam mixture, which is further heated in the convection section. The weight ratio of dilution steam to the hydrocarbon feed can range from, e.g., 0.2 to 0.6, preferably from 0.25 to 0.4. The further heated feed-steam mixture then enters into the cross-over section 109 of the tubing, which, as shown, can be located outside of the furnace housing. The heated feed-steam mixture in the cross-over section 109 can have a temperature ranging from, e.g., 690 to 780° C., preferably from 760 to 775° C. In certain embodiments, it is desirable that cracking of some hydrocarbon molecules occur in the cross-over section to a certain extent.

At the end of the cross-over section, the heated feed-steam mixture then enters into one or more radiant tube 111 located in the radiant section 113 of the steam cracking furnace, which are typically heated by the thermal energy released by combusting a fuel at a plurality of burners. In the radiant tube, the feed-steam mixture is heated to a high pyrolysis temperature, e.g., from 800 to 920° C., preferably from 800 to 840° C., for a short residence time ranging from, e.g., 0.1 to 1.0 second, preferably from 0.1 to 0.3 second, to effect cracking of hydrocarbon molecules to produce a radiant effluent comprising desired olefin molecules and byproducts exiting the radiant tube. For example, ethane in the feed desirably undergo the following reactions:

C₂H₆←→H₂C═CH₂+H₂

The above forward, cracking reaction is highly endothermic, thus a higher temperature is conducive to the conversions of alkanes to produce the desired olefins. In addition, the above forward reactions result in increase of moles of gaseous species, and thus a lower pressure inside the radiant tube, and hence at the outlet of the radiant tube (coil outlet pressure, “COP”) is conducive to the conversions of ethane to produce ethylene. Thus, in order to achieve high alkane conversions and a high selectivity to desired olefins, in conventional steam cracking furnaces, the radiant tubes are operated under conditions such that the COP is generally lower than 170 kPa-gauge.

The radiant effluent exiting the radiant tubes is typically cooled by direct and/or indirect cooling using, e.g., a transfer line exchanger (“TLE”) and/or quench oil. The thus cooled effluent is then separated to produce a process gas rich in hydrogen and C1-C4 hydrocarbons including the desirable olefin molecules. Efficient and effective separation of the process gas in the recovery section of an olefins production plant based on steam cracking requires the compression of the process gas to a pressure as high as 4200 kPa-gauge, and cooling the process gas or separated components therein to temperatures as low as −160° C. The compression of the process gas to such elevated pressure from a COP less than 200 kPa-gauge requires using at least four stages of compressors. In a modern olefins production plant, the typical power rating of any of such compressors can be as high as 75 MW depending on production rate. The costs of purchasing, installing, operating, and maintaining such multiple compressors are very high. As such, it would be highly desirable to eliminate one or more stages of the compressors for a modern steam cracking-based olefins production plant.

Increasing the COP has the potential to eliminate one or more of the multiple stages of process gas compressors otherwise required in a conventional olefins production plant. However, as discussed above, increasing COP while all other conditions held equal would decrease ethane conversion to ethylene. To compensate, one can raise the pyrolysis temperature and/or increase residence time in the radiant tube(s). That can result in a decreased selectivity toward the desired olefins products and an increased production of low-value byproducts such as coke, which can cause fouling in the radiant tube and downstream equipment such as the TLE and the quench tower. Thus, it is a complicated and very difficult undertaking to reduce one or more stages of compressors in a steam-cracker-based olefins production plant.

The present inventors have invented new processes with unexpected advantages capable of reducing the total number of stages of process gas compressors to three or fewer by implementing a steam cracking process with a COP from 200 kPa-gauge to 700 kPa-gauge, e.g., from 200, 210, 220, 230, 240, 250, 260, 270, 280, 290, 300 kPa-gauge to 320, 340, 350, 360, 380, 400 kPa-gauge, to 420, 440, 450, 460, 470, 480, 500 kPa-gauge, to 520, 540, 550, 560, 580, 600 kPa-gauge, to 620, 640, 650, 660, 680, 700 kPa-gauge.

To compensate the reduced alkane conversion due to higher COP, a longer residence time in the radiant can be used in the processes of this disclosure than in conventional steam cracking processes with a low COP of lower than 200 kPa-gauge. This, however, can result in the production of more byproducts such as coke in the radiant tube.

To manage the resulting higher production of coke, the radiant tube can desirably comprise a cast body of a heat-resistant alloy consisting essentially of, in mass percent, 0.05 to 0.7% of C, more than 0% and no more than 2.5% of Si, more than 0% and no more than 3.0% of Mn, 15 to 50% of Cr, 20 to 70% of Ni, 2 to 4% of Al, 0.005 to 0.4% of rare-earth elements, and at least one member selected from the group consisting of 0.5 to 10% of W and 0.1 to 5% of Mo, the balance being Fe and inevitable impurities. A barrier layer can be formed on a surface of the cast body contacting the heated feed-steam mixture (i.e., the inner surface of the radiant tube), wherein the barrier layer can comprise an Al₂O₃ layer having a thickness of 0.5 um or more. The Al₂O₃ barrier layer can be formed in situ during the operation the radiant tubes under the pyrolysis reaction conditions. At least 80 area % of an outermost surface of the cast body can be Al₂O₃. The base body can have Cr-based particles dispersed at an interface between the Al₂O₃ layer and the cast body at a higher Cr concentration than a Cr concentration of a matrix of the alloy. The surface of the cast body (i.e., the inner surface of the radiant tubes) before forming Al₂O₃ layer can have of a roughness (Ra) of 0.05 to 2.5. It has been found that the barrier layer is particularly resistant to coke/tar deposited thereon, functioning to extend the life of the radiant tube in a higher temperature environment prone to forming higher amount of coke. In addition the presence of the stabilized Al₂O₃ layer gives the cast product outstanding cyclic oxidation resistance, carburization resistance, nitriding resistance and corrosion resistance over a prolonged period of time of use in high temperature condition.

Referring to the FIGURE, the radiant effluent 115 exiting the steam cracking furnace is immediately cooled at a direct or indirect heat exchanger 117 (e.g., a TLE, and/or a quench exchanger) to produce a cooled effluent stream 119 to stop undesirable chemical reactions in the effluent at elevated temperatures. The cooled effluent stream 119 can be then fed into a feed stream inlet of a coke-separation device 121 (e.g., a coke catchleg, and/or a centrifuge) to obtain a coke-abated cooled effluent stream 125 exiting a vapor outlet of the coke-separation device, which is a coke-depleted vapor stream, and deposited coke from a coke particle deposition section of the coke-separation device 121. The coke-abated cooled effluent stream 125 may nonetheless comprise a non-negligible amount of coke. By using the coke-separation device 121, a significant portion of coke and/or tar in the cooled radiant effluent stream 119 can be removed, resulting in reduced amount of coke and/or tar introduced into downstream equipment such as the quench tower and quench separation drum, and significantly reduced likelihood of fouling in these downstream equipment caused by coke and/or tar. This can allow for a higher pyrolysis temperature in the radiant tube as described above which can increase alkane conversion while producing increased amount of coke and steam cracker tar. This device can also allow for additional coke/tar load to the system from operations such as online decoking, thus increasing furnace availability. In addition, it may decrease the sizing of the quench tower bottoms and the first compartment of the quench tower drum since these are designed based on coke/tar load.

As shown in the FIGURE, stream 125 is then fed into a water quench tower 127, which also receives a quench water stream 159. Inside tower 127, materials in stream 125 contacts the quench water, are further cooled, and separated to produce a quench water tower overhead stream 129 at a location in the vicinity of the top, preferably at the top, of tower 127. Stream 129 comprises H₂. C1-C4 hydrocarbons, steam, optionally a small quantity of C5+ hydrocarbons, and optionally various quantities of acid gases (CO₂, H₂S, and the like). Stream 129 can have a pressure similar to the COP, e.g., from 200, 210, 220, 230, 240, 250, 260, 270, 280, 290, 300 kPa-gauge to 320, 340, 350, 360, 380, 400 kPa-gauge, to 420, 440, 450, 460, 470, 480, 500 kPa-gauge, to 520, 540, 550, 560, 580, 600 kPa-gauge, to 620, 640, 650, 660, 680, 700 kPa-gauge. Upon optional condensing/drying, stream 129 (process gas stream) can be fed into the first stage compressor 131 to produce a first compressed process gas stream 133 having a pressure higher than COP ranging from, e.g., 310 kPa-gauge to 960 kPa-gauge. Upon cooling and optional separation and/or treatment (not shown), stream 133, or a portion or a fraction thereof, may be fed into a second stage compressor 135 to produce a twice-compressed stream 137 having a pressure higher than stream 133 ranging from, e.g., 510 kPa-gauge to 1830 kPa-gauge. Upon cooling and optional separation and/or treatment (not shown), stream 137, or a portion or a fraction thereof, may be fed into a third stage compressor 139 to produce a thrice-compressed stream 141 having a pressure higher than stream 137 ranging from, e.g., 930 kPa-gauge to 4200 kPa-gauge. Stream 141 can be further subjected to cooling, separation, and/or treatment (not shown). Non-limiting examples of such optional treatment include: drying, sour gas washing, selective alkyne and diene hydrogenation, and the like, as is known in conventional olefins production plants. The compression, cooling, separation and treatment of streams 133, 137, and 141 can produce various products and by-products in the recovery section of an olefins production plant, such as ethylene, propylene, butenes, butadiene, hydrogen, tail gas, recycle ethane stream, sour gas stream, a C5+ hydrocarbon stream, and the like. As a result of the high COP in the processes of this disclosure, no more than three stages of compressors may be required in the recovery section to recover the desired olefins products. Given the high power rating and high associated costs of each stage of process gas compressor, compared to conventional steam cracking processes having a COP lower than 200 kPa-gauge necessitating four or more stages of process gas compressors, this high COP feature of the processes of this disclosure translates into significant savings in procurement, installation, operation, and maintenance of the additional stage(s) of compressor(s).

From the vicinity of the bottom, including but not limited to the bottom, of quench tower 127, a first quench tower liquid effluent stream 144 is produced. In certain embodiments (not shown), stream 144 may be drawn as a side stream from a lower location above the bottom of tower 127 in the vicinity of the bottom of tower 127. In such embodiments, a second quench tower liquid effluent stream (not shown) may be drawn from the bottom of tower 127, which can comprise coke at a higher quantity or concentration than in the first quench tower liquid effluent stream 144. In such embodiments, it may be desirable to feed a tar solvent, continuously or intermittently, into the second quench tower liquid effluent stream in order to prevent and/or mitigate fouling in the line conducting away the second quench tower liquid effluent stream, and/or downstream process/equipment, as described below.

In certain other embodiments, as shown in the FIGURE, the first quench tower liquid effluent stream 144 can be drawn from the bottom of tower 127. Regardless of the location at which stream 144 is drawn, stream 144 comprises water, coke/tar, steam cracker naphtha, steam cracker gas oil, and coke, at various quantities.

Though not shown, additional streams separate from stream 144 in the vicinity of the bottom of, in the middle of, and/or in the vicinity of the top of, tower 127 may be produced as well.

Stream 144, upon optionally continuously or intermittently mixing with a tar solvent stream 185, such as a stream comprising C6-C7 aromatics as described later, if needed, to form a stream 145 to reduce fouling of the pipe and downstream equipment (e.g., the quench separation drum 147), is then fed into the quench separation drum 147. From drum 147, a coke-rich stream 148, a first aqueous stream 151, an optional second aqueous stream 161, and an oil stream 177 are produced. Stream 148, upon optionally mixing with a tar solvent stream 187, such as a stream comprising C6-C7 aromatics described later, if needed, to form a stream 149, can be then fed into the coke-separation device 121 to produce a portion of coke product 123.

The first aqueous stream 151, optionally upon mixing with a tar solvent stream 191, such as a stream comprising C6-C7 aromatics described later, if needed, to form a stream 152, can be then fed into a coke-removing device 153, to produce a coke-depleted water stream 155. Device 153 can be or can include, e.g., a filter. The coke-depleted water stream 155, upon cooling via a heat exchanger 157, can be advantageously used as a portion, or the entirety of the quench water stream 159 fed into the quench tower 127 as described above. By further removing coke from stream 152 with coke-removing device 153, the coke-depleted water stream 155 can find beneficial use in a petrochemical plant or can be supplied to a wastewater treatment plant, as the case may be. Preferably, a part or the entirety of stream 155 is recycled to the quench tower 127 as at least a portion of the quench water stream 159. The reduced coke loading in stream 155 reduces the probability of coke-induced fouling in quench tower 127.

The optional second aqueous stream 161, upon optional mixing with a tar solvent stream 189, such as a stream comprising C6-C7 aromatics as described later, if needed, to form a stream 162, can be then fed into a stripping column 163. Column 163 is advantageously operated at a bottoms temperature no greater than 280° F. (135° C.), preferably no greater than 250° F., and an overhead pressure no greater than 250 kPa-gauge, preferably no greater than 200 kPa-gauge, e.g., ranging from 27, 30, 40, 50, 60, 70, 80, 90, 100 kPa-gauge, to 120, 140, 150, 160, 180, 200, 220, 240, 250 kPa-gauge. Probability of fouling in the stripping column caused by, e.g., styrene present in stream 161, can be too high if the bottoms temperature exceeds 280° F. (121° C.). To achieve the low bottoms temperature, a relatively low overheads pressure of no greater than 250 kPa-gauge is highly desired. From column 163, an overheads stream 169 comprising steam, sour gases (e.g., CO₂, H₂S), and naphtha boiling range hydrocarbons, and a bottoms stream 165 consisting essentially of water are produced. A steam stream 167 may be fed into the stripping column 163 to facilitate the separation of sour gases and naphtha-range hydrocarbons into the overheads stream 169. Alternatively, the stripping steam may be generated in situ, in part or in whole, by using a reboiler (not shown). Stream 165, depleted with sour gas, can be advantageously supplied to a dilution steam generator to generate dilution steam for stream 105. Overhead stream 169, if having a pressure no greater than 200 kPa-gauge, can be converted into a higher-pressure stripping column recycle stream 175 by device 173 and then recycled to quench tower 127. In a particularly advantageous embodiment, device 173 can be or can include an ejector that receives stream 169 as a low-pressure feed stream and a motive stream 171 (e.g., steam) to produce a combined stream 175 having a higher pressure than stream 169, suitable for recycling into tower 127. Alternatively, device 173 can be or can include a compressor. By using device 173, one can operate the stripping tower 163 at a desired low overhead pressure of no greater than 250 kPa-gauge to prevent fouling therein, while also conveniently recycling the overhead stream 169 into the quench tower 127.

The oil stream 177 exiting the quench separation drum 147 can be separated by using one or more separation devices including but not limited to distillation columns 179 to produce, e.g., a C4-C5 stream 181, a C6-C7 aromatics-rich stream 183, and a heavy fuel oil stream 185. Stream 183 can be advantageously used as a tar solvent. Thus, as shown and as described above, steam 183 can be split into one or more split streams 185, 187, 189, and 191, which can be mixed with one or more of streams 144, 148, 161, and 151, respectively or as needed, to produce one or more of corresponding combined streams 145, 149, 162, and 152, respectively, in order to mitigate the impact of any coke/tar that may be present in streams 144, 148, 161, and 151, respectively. While any other C6-C7 aromatics-rich stream imported from external sources to the processes of this disclosure may be used as tar solvent to treat any of streams 144, 148, 161, and 151, the in-situ derived stream 183 separated from stream 177, is particularly advantageous for this purpose given that stream 177 comprises C6-C7 aromatics, and a C6-C7 aromatics-rich stream with sufficient quantity and quality requirements can be conveniently separated from stream 177 at a low cost.

This disclosure can include the following non-limiting aspects and/or embodiments.

A1. A process for producing a C2-C4 olefin product from an ethane-containing hydrocarbon feed, the process comprising:
(I) providing the ethane-containing hydrocarbon feed;
(II) mixing the ethane-containing hydrocarbon feed with a dilution steam to produce a feed-steam mixture;
(III) heating the feed-steam mixture in a convection section of a steam cracking furnace to obtain a heated feed-steam mixture;
(IV) cracking the heated feed-steam mixture in a radiant tube located in a radiant section of the steam cracking furnace under pyrolysis conditions to produce a radiant effluent exiting the steam cracking furnace having a coil-outlet pressure (“COP”) of from 200 kPa-gauge to 700 kPa-gauge;
(V) cooling the radiant effluent to obtain a cooled effluent;
(VI) feeding at least a portion of the cooled effluent into a quench tower;
(VII) feeding a quench water stream into the quench tower to contact the cooled effluent in the quench tower;
(VIII) obtaining a quench tower overhead vapor stream at a location in the vicinity of the top of the quench tower, and a first quench tower liquid effluent stream at a location in the vicinity of the bottom of the quench tower, from the quench tower;
(IX) recovering the C2-C4 olefin product from the quench tower overhead vapor stream by using no more than 3 stages of compression;
(X) separating the first quench tower liquid effluent stream optionally mixed with a tar solvent, to obtain a coke-rich stream, a first aqueous stream, an optional second aqueous stream, and an oil stream;
(XI) removing at least a portion of the coke contained in the first aqueous stream to obtain a coke-depleted water stream; and
(XII) supplying the coke-depleted water stream as at least a portion of the quench water stream in step (VII).

A2. The process of A1, wherein in step (VIII), the first quench tower liquid effluent stream is obtained from the bottom of the quench tower.

A3. The process of A1 or A2, wherein in step (VIII), the first quench tower liquid effluent stream is obtained from a location above the bottom of the quench tower, and a second quench tower liquid effluent stream is obtained from the bottom of the quench tower.

A4. The process of any of A1 to A3, further comprising at least one of the following:
(XIII) mixing the coke-rich stream with a tar solvent;
(XIV) mixing the first aqueous stream with a tar solvent;
(XV) mixing the second aqueous stream with a tar solvent; and
(XVI) mixing the second quench tower liquid effluent stream with a tar solvent.

A5. The process of any of A1 to A4, wherein step (VI) comprises:
(VIa) removing at least a portion of the coke contained in the cooled effluent to obtain a coke-abated cooled effluent stream; and
(VIb) feeding the coke-abated cooled effluent stream as the portion of the cooled effluent into the quench tower.

A6. The process of A5, wherein step (VIa) comprises:
(VIa.1) providing a separation device comprising a feed stream inlet, a vapor outlet, and a coke particle deposition section;
(VIa.2) feeding the cooled effluent into the feed stream inlet;
(VIa.3) obtaining a coke-depleted vapor stream from the vapor outlet as the coke-abated cooled effluent stream; and
(VIa.4) obtaining deposited coke from the coke deposition section.

A7. The process of any of A1 to A6, wherein in step (X), the second aqueous stream is obtained, and the process further comprises:
(XIX) stripping the second aqueous stream in a stripping column at a column bottom temperature of no greater than 280° F. and a stripping column overhead pressure of no greater than 250 kPa-gauge to obtain a stripping column overhead vapor stream and a stripping column bottoms process water stream; and
(XX) forming a stripping column recycle stream having a higher pressure than the stripping column overhead vapor stream from the stripping column overhead vapor stream; and
(XXI) feeding the stripping column recycle stream into the quench tower.

A8. The process of A7, wherein step (XVII) is carried out at a stripping column overhead pressure from 27 kPa-gauge to 96 kPa-gauge.

A9. The process of A7 or A8, further comprising:
(XXII) feeding at least a portion of the stripping column bottoms process water stream into a steam generator; and
(XXIII) generating steam from the dilution steam generator.

A10. The process of any of A1 to A9, further comprising:
(XVII) separating from at least the oil stream a C6-C7 aromatics-rich stream; and
(XVIII) supplying at least a portion of the C6-C7 aromatics-rich stream as at least a portion of the tar solvent in at least one of steps (X), (XIII), (XIV), (XV), and (XVI).

A11. The process of A9 or A10, wherein at least a portion of the steam generated in step (XVIII) is used as at least portion of the dilution steam of step (II).

A12. The process of any of A7 to A11, wherein step (XX) comprises at least one of the following:
(XXa) feeding the stripping column overhead vapor stream and an ejector motive stream into an ejector to obtain the stripping column recycle stream exiting the ejector; and
(XXb) compressing the stripping column overhead vapor stream to obtain the stripping column recycle stream.

A13. The process of any of A1 to A12, wherein in step (IV), the radiant tube comprises:
a cast body of a heat-resistant alloy consisting essentially of, in mass percent, 0.05 to 0.7% of C, more than 0% and no more than 2.5% of Si, more than 0% and no more than 3.0% of Mn, 15 to 50% of Cr, 20 to 70% of Ni, 2 to 4% of Al, 0.005 to 0.4% of rare-earth elements, and at least one member selected from the group consisting of 0.5 to 10% of W and 0.1 to 5% of Mo, the balance being Fe and inevitable impurities; and
a barrier layer formed on a surface of the cast body to be brought into contact with the heated feed-steam mixture;
wherein preferably the barrier layer comprises an Al₂O₃ layer having a thickness of 0.5 μm or more;
wherein preferably at least 80 area % of an outermost surface of the cast body is Al₂O₃;
wherein preferably the cast body has Cr-based particles dispersed at an interface between the Al₂O₃ layer and the cast body at a higher Cr concentration than a Cr concentration of a matrix of the alloy; and
wherein preferably the surface of the cast body before forming Al₂O₃ layer is of a roughness (Ra) of 0.05 to 2.5.

A14. The process of any of A1 to A13, wherein the heated feed-steam mixture of step (III) is supplied to the radiant tube in step (IV) through a cross-over pipe, and the heated feed-steam mixture in the cross-over pipe has a temperature in a range from 691° C. to 777° C.

A14. The process of any of A1 to A13, and hydrocarbon cracking occurs in the cross-over pipe.

A15. The process of any of A1 to A14, wherein the ethane-containing hydrocarbon feed comprises ethane at a concentration of at least 50 mol %, based on the total moles of hydrocarbons therein.

A16. The process of any of A1 to A15, wherein:
in step (IX), three sequential stages of compression are used, consisting of a first stage, a second stage downstream of the first stage, and a third stage downstream of the second stage;
the first stage has a first stage outlet pressure in a range from 310 kPa-gauge to 960 kPa-gauge;
the second stage has a second stage outlet pressure in a range from 510 kPa-gauge to 1830 kPa-gauge that is higher than the first stage outlet pressure; and
the third stage has a third stage outlet pressure in a range from 930 kPa-gauge to 4200 kPa-gauge that is higher than the second stage outlet pressure.

B1. A process for producing a C2-C4 olefin product from an ethane-containing hydrocarbon feed, the process comprising:
(1) providing the ethane-containing hydrocarbon feed;
(2) mixing the ethane-containing hydrocarbon feed with a dilution steam to produce a feed-steam mixture;
(3) heating the feed-steam mixture in a convection section of a steam cracking furnace to obtain a heated feed-steam mixture;
(4) cracking the heated feed-steam mixture in a radiant tube located in a radiant section of the steam cracking furnace under pyrolysis conditions to produce a radiant effluent exiting the cracking furnace having a coil-outlet pressure (“COP”) of from 200 kPa-gauge to 700 kPa-gauge;
(5) cooling the radiant effluent to obtain a cooled effluent;
(6) feeding at least a portion of the cooled effluent into a quench tower;
(7) feeding a quench water stream into the quench tower to contact the cooled effluent in the quench tower;
(8) obtaining a quench tower overhead vapor stream at a location in the vicinity of the top of the quench tower, and a first quench tower liquid effluent stream at a location in the vicinity of the bottom of the quench tower, from the quench tower;
(9) processing the quench tower overhead vapor stream to obtain the C2-C4 olefin product by using no more than 3 stages of compression;
(10) separating the first quench tower liquid effluent stream optionally mixed with a tar solvent, to obtain a coke-rich stream, a first aqueous stream, a second aqueous stream, and an oil stream; and
(11) stripping the second aqueous stream in a stripping column at a column bottom temperature of no greater than 280° F. and a stripping column overhead pressure of no greater than 250 kPa-gauge to obtain a stripping column overhead vapor stream and a stripping column bottoms process water stream; and
(12) forming a stripping column recycle stream having a higher pressure than the stripping column overhead vapor stream from the stripping column overhead vapor stream; and
(13) feeding the stripping column recycle stream into the quench tower.

B2. The process of B1, wherein step (12) comprises at least one of the following:
(13a) feeding the stripping column overhead vapor stream and an ejector motive stream into an ejector to obtain the stripping column recycle stream exiting the ejector; and
(13b) compressing the stripping column overhead vapor stream to obtain the stripping column recycle stream.

B3. The process of B1 or B2, further comprising:
(14) feeding at least a portion of the stripping column bottoms process water stream into a dilution steam generator; and
(15) generating at least a portion of the dilution stream of step (2) from the dilution steam generator.

B4. The process of any of B1 to B3, further comprising:
(16) removing at least a portion of the coke contained in the first aqueous stream to obtain a coke-depleted water stream; and
(17) supplying the coke-depleted water stream as at least a portion of the quench water stream in step (7).

B5. The process of any of BI to B4, wherein in step (8), the first quench tower liquid effluent stream is obtained from the bottom of the quench tower.

B6. The process of any of B1 to B4, wherein in step (8), the first quench tower liquid effluent stream is obtained from a location above the bottom of the quench tower, and a second quench tower liquid effluent stream is obtained from the bottom of the quench tower.

B7. The process of any of B1 to B7, further comprising at least one of the following:
(18) mixing the coke-rich stream with a tar solvent;
(19) mixing the first aqueous stream with a tar solvent;
(20) mixing the second aqueous stream with a tar solvent; and
(21) mixing the second quench tower liquid effluent stream with a tar solvent.

B8. The process of any of B1 to B7, wherein step (6) comprises:
(6a) removing at least a portion of the coke contained in the cooled effluent to obtain a coke-abated cooled effluent stream; and
(6b) feeding the coke-abated cooled effluent stream as the portion of the cooled effluent into the quench tower.

B9. The process of B8, wherein step (6a) comprises:
(6a.1) providing a separation device comprising a feed stream inlet, a vapor outlet, and a coke particle deposition section;
(6a.2) feeding the cooled effluent into the feed stream inlet;
(6a.3) obtaining a coke-depleted vapor stream from the vapor outlet as the coke-abated cooled effluent stream; and
(6a.4) obtaining deposited coke from the coke deposition section.

B10. The process of any of B1 to B9, wherein step (11) is carried out at a stripping column overhead pressure from 27 kPa-gauge to 96 kPa-gauge.

B11. The process of any of B1 to B10, further comprising:
(22) separating from at least the oil stream a C6-C7 aromatics-rich stream; and
(23) supplying at least a portion of the C6-C7 aromatics-rich stream as at least a portion of the tar solvent in at least one of steps (10), (18), (19), (20), and (21).

B12. The process of any of B1 to B11, wherein in step (4), the radiant tube comprises:
a cast body of a heat-resistant alloy consisting essentially of, in mass percent, 0.05 to 0.7% of C, more than 0% and no more than 2.5% of Si, more than 0% and no more than 3.0% of Mn, 15 to 50% of Cr, 20 to 70% of Ni, 2 to 4% of Al, 0.005 to 0.4% of rare-earth elements, and at least one member selected from the group consisting of 0.5 to 10% of W and 0.1 to 5% of Mo, the balance being Fe and inevitable impurities; and
a barrier layer formed on a surface of the cast body to be brought into contact with the heated feed-steam mixture;
wherein preferably the barrier layer comprises an Al₂O₃ layer having a thickness of 0.5 μm or more;
wherein preferably at least 80 area % of an outermost surface of the cast body is Al₂O₃;
wherein preferably the cast body has Cr-based particles dispersed at an interface between the Al₂O₃ layer and the cast body at a higher Cr concentration than a Cr concentration of a matrix of the alloy; and
wherein preferably the surface of the cast body before forming Al₂O₃ layer is of a roughness (Ra) of 0.05 to 2.5.

B13. The process of any of B1 to B12, wherein the heated feed-steam mixture of step (3) is supplied to the radiant tube in step (4) through a cross-over pipe, and the heated feed-steam mixture in the cross-over pipe has a temperature in a range from 691° C. to 777° C.

B14. The process of any of B1 to B13, and hydrocarbon cracking occurs in the cross-over pipe.

B15. The process of any of B1 to B14, wherein the ethane-containing hydrocarbon feed comprises ethane at a concentration of at least 50 mol %, based on the total moles of hydrocarbons therein.

B16. The process of any of B1 to B15, wherein:
in step (9), three sequential stages of compression are used, consisting of a first stage, a second stage downstream of the first stage, and a third stage downstream of the second stage;
the first stage has a first stage outlet pressure in a range from 310 kPa-gauge to 960 kPa-gauge;
the second stage has a second stage outlet pressure in a range from 510 kPa-gauge to 1830 kPa-gauge that is higher than the first stage outlet pressure; and
the third stage has a third stage outlet pressure in a range from 930 kPa-gauge to 4200 kPa-gauge that is higher than the second stage outlet pressure.

C1. A process for producing a C2-C4 olefin product from an ethane-containing hydrocarbon feed, the process comprising:
(a) providing the ethane-containing hydrocarbon feed;
(b) mixing the ethane-containing hydrocarbon feed with a dilution steam to produce a feed-steam mixture;
(c) heating the feed-steam mixture in a convection section of a steam cracking furnace to obtain a heated feed-steam mixture;
(d) cracking the heated feed-steam mixture in a radiant tube located in a radiant section of the steam cracking furnace under pyrolysis conditions to product a radiant effluent exiting the cracking furnace having a coil-outlet pressure (“COP”) of from 200 kPa-gauge to 700 kPa-gauge;
(e) cooling the radiant effluent to obtain a cooled effluent;
(f) feeding at least a portion of the cooled effluent into a quench tower by:
   (f.1) providing a separation device comprising a feed stream inlet, a vapor outlet, and a coke particle deposition section;
   (f.2) feeding the cooled effluent into the feed stream inlet;
   (f.3) obtaining a coke-depleted vapor stream from the vapor outlet as the coke-abated cooled effluent stream; and
   (f.4) obtaining deposited coke from the coke deposition section;
(g) feeding a quench water stream into the quench tower to contact the cooled effluent in the quench tower;
(h) obtaining a quench tower overhead vapor stream at a location in the vicinity of the top of the quench tower, and a first quench tower liquid effluent stream at a location in the vicinity of the bottom of the quench tower, from the quench tower;
(i) processing the quench tower overhead vapor stream to obtain the C2-C4 olefin product by using no more than 3 stages of compression; and
(j) separating the first quench tower liquid effluent stream optionally mixed with a tar solvent, to obtain a coke-rich stream, a first aqueous stream, a second aqueous stream, and an oil stream.

C2. The process of C1, further comprising:
(k) feeding at least a portion of the coke-rich stream into the coke-separation device of step (f.1).

C3. The process of C1 or C2, further comprising:
(1) stripping the second aqueous stream in a stripping column at a column bottom temperature of no greater than 280° F. and a stripping column overhead pressure of no greater than 250 kPa-gauge to obtain a stripping column overhead vapor stream and a stripping column bottoms process water stream; and
(m) forming a stripping column recycle stream having a higher pressure than the stripping column overhead vapor stream from the stripping column overhead vapor stream; and
(o) feeding the stripping column recycle stream into the quench tower.

C4. The process of C3, wherein step (m) comprises at least one of the following:
(m1) feeding the stripping column overhead vapor stream and an ejector motive stream into an ejector to obtain the stripping column recycle stream exiting the ejector; and
(m2) compressing the stripping column overhead vapor stream to obtain the stripping column recycle stream.

C5. The process of C3, further comprising:
(p) feeding at least a portion of the stripping column bottoms process water stream into a dilution steam generator; and
(q) generating at least a portion of the dilution stream of step (b) from the dilution steam generator.

C6. The process of any of C1 to C5, wherein in step (g), the first quench tower liquid effluent stream is obtained from the bottom of the quench tower.

C7. The process of any of C1 to C5, wherein in step (g), the first quench tower liquid effluent stream is obtained from a location above the bottom of the quench tower, and a second quench tower liquid effluent stream is obtained from the bottom of the quench tower.

C8. The process of any of C1 to C7, further comprising at least one of the following:
(r) mixing the coke-rich stream with a tar solvent;
(s) mixing the first aqueous stream with a tar solvent;
(t) mixing the second aqueous stream with a tar solvent; and
(u) mixing the second quench tower liquid effluent stream with a tar solvent.

C9. The process of any of C3 to C8, wherein step (1) is carried out at a stripping column overhead pressure from 27 kPa-gauge to 96 kPa-gauge.

C10. The process of any of C1 to C9, further comprising:
(v) separating from at least the oil stream a C6-C7 aromatics-rich stream; and
(w) supplying at least a portion of the C6-C7 aromatics-rich stream as at least a portion of the tar solvent in at least one of steps (j), (r), (s), (t), and (u).

C11. The process of any of C1 to C10, wherein in step (d), the radiant tube comprises:
a cast body of a heat-resistant alloy consisting essentially of, in mass percent, 0.05 to 0.7% of C, more than 0% and no more than 2.5% of Si, more than 0% and no more than 3.0% of Mn, 15 to 50% of Cr, 20 to 70% of Ni, 2 to 4% of Al, 0.005 to 0.4% of rare-earth elements, and at least one member selected from the group consisting of 0.5 to 10% of W and 0.1 to 5% of Mo, the balance being Fe and inevitable impurities; and
a barrier layer formed on a surface of the cast body to be brought into contact with the heated feed-steam mixture;
wherein preferably the barrier layer comprises an Al2O3 layer having a thickness of 0.5 μm or more;
wherein preferably at least 80 area % of an outermost surface of the cast body is Al₂O₃;
wherein preferably the cast body has Cr-based particles dispersed at an interface between the Al₂O₃ layer and the cast body at a higher Cr concentration than a Cr concentration of a matrix of the alloy; and
wherein preferably the surface of the cast body before forming Al₂O₃ layer is of a roughness (Ra) of 0.05 to 2.5.

C12. The process of any of C1 to C11, wherein the heated feed-steam mixture of step (c) is supplied to the radiant tube in step (d) through a cross-over pipe, and the heated feed-steam mixture in the cross-over pipe has a temperature in a range from 691° C. to 777° C.

C13. The process of any of C1 to C12, and hydrocarbon cracking occurs in the cross-over pipe.

C14. The process of any of C1 to C13, wherein the ethane-containing hydrocarbon feed comprises ethane at a concentration of at least 50 mol %, based on the total moles of hydrocarbons therein.

C15. The process of any of C1 to C14, wherein:
in step (i), three sequential stages of compression are used, consisting of a first stage, a second stage downstream of the first stage, and a third stage downstream of the second stage;
the first stage has a first stage outlet pressure in a range from 310 kPa-gauge to 960 kPa-gauge;
the second stage has a second stage outlet pressure in a range from 510 kPa-gauge to 1830 kPa-gauge that is higher than the first stage outlet pressure; and
the third stage has a third stage outlet pressure in a range from 930 kPa-gauge to 4200 kPa-gauge that is higher than the second stage outlet pressure.

D1. A process for producing a C2-C4 olefin product from an ethane-containing hydrocarbon feed, the process comprising:
(A) providing the ethane-containing hydrocarbon feed;
(B) mixing the ethane-containing hydrocarbon feed with a dilution steam to produce a feed-steam mixture;
(C) heating the feed-steam mixture in a convection section of a steam cracking furnace to obtain a heated feed-steam mixture;
(D) cracking the heated feed-steam mixture in a radiant tube located in a radiant section of the steam cracking furnace under pyrolysis conditions to produce a radiant effluent exiting the cracking furnace having a coil-outlet pressure (“COP”) of from 200 kPa-gauge to 700 kPa-gauge;
(E) cooling the radiant effluent to obtain a cooled effluent;
(F) feeding at least a portion of the cooled effluent into a quench tower;
(G) feeding a quench water stream into the quench tower to contact the cooled effluent in the quench tower;
(H) obtaining a quench tower overhead vapor stream at a location in the vicinity of the top of the quench tower, and a first quench tower liquid effluent stream at a location in the vicinity of the bottom of the quench tower, from the quench tower;
(I) processing the quench tower overhead vapor stream to obtain the C2-C4 olefin product by using no more than 3 stages of compression;
(J) separating the first quench tower liquid effluent stream optionally mixed with a tar solvent, to obtain a coke-rich stream, a first aqueous stream, a second aqueous stream, and an oil stream; and
(K) separating from the oil stream a C6-C7 aromatics-rich stream; and
(L) mixing at least a portion of the C6-C7 aromatics-rich stream with at least one of the following: (i) the first quench tower liquid effluent stream as at least a portion of the tar solvent in step (J); (ii) the coke-rich stream after step (J); (iii) the first and/or second aqueous streams after step (J).

D2. The process of D1, further comprising:
(M) stripping the second aqueous stream in a stripping column at a column bottom temperature of no greater than 280° F. and a stripping column overhead pressure of no greater than 250 kPa-gauge to obtain a stripping column overhead vapor stream and a stripping column bottoms process water stream; and
(N) forming a stripping column recycle stream having a higher pressure than the stripping column overhead vapor stream from the stripping column overhead vapor stream; and
(O) feeding the stripping column recycle stream into the quench tower.

D3. The process of D2, wherein step (N) comprises at least one of the following:
(N1) feeding the stripping column overhead vapor stream and an ejector motive stream into an ejector to obtain the stripping column recycle stream exiting the ejector; and
(N2) compressing the stripping column overhead vapor stream to obtain the stripping column recycle stream.

D4. The process of D2 or D3, further comprising:
(P) feeding at least a portion of the stripping column bottoms process water stream into a dilution steam generator; and
(Q) generating at least a portion of the dilution stream of step (B) from the dilution steam generator.

D5. The process of any of D1 to D4, further comprising:
(R) removing at least a portion of the coke contained in the first aqueous stream to obtain a coke-depleted water stream; and
(S) supplying the coke-depleted water stream as at least a portion of the quench water stream in step (G).

D6. The process of any of D1 to D5, wherein in step (H), the first quench tower liquid effluent stream is obtained from the bottom of the quench tower.

D7. The process of any of D1 to D5, wherein in step (H), the first quench tower liquid effluent stream is obtained from a location above the bottom of the quench tower, and a second quench tower liquid effluent stream is obtained from the bottom of the quench tower.

D8. The process of any of D1 to D7, further comprising at least one of the following:
(T) mixing the coke-rich stream with a tar solvent;
(U) mixing the first aqueous stream with a tar solvent;
(V) mixing the second aqueous stream with a tar solvent; and
(W) mixing the second quench tower liquid effluent stream with a tar solvent.

D9. The process of any of D1 to D8, wherein step (F) comprises:
(F1) removing at least a portion of the coke contained in the cooled effluent to obtain a coke-abated cooled effluent stream; and
(F2) feeding the coke-abated cooled effluent stream as the portion of the cooled effluent into the quench tower.

D10. The process of D9, wherein step (F1) comprises:
(F1.a) providing a separation device comprising a feed stream inlet, a vapor outlet, and a coke particle deposition section;
(F1.b) feeding the cooled effluent into the feed stream inlet;
(F1.c) obtaining a coke-depleted vapor stream from the vapor outlet as the coke-abated cooled effluent stream; and
(F1.d) obtaining deposited coke from the coke deposition section.

D11. The process of any of D2 to D10, wherein step (M) is carried out at a stripping column overhead pressure from 27 kPa-gauge to 96 kPa-gauge.

D12. The process of any of D1 to D11, wherein in step (D), the radiant tube comprises:
a cast body of a heat-resistant alloy consisting essentially of, in mass percent, 0.05 to 0.7% of C, more than 0% and no more than 2.5% of Si, more than 0% and no more than 3.0% of Mn, 15 to 50% of Cr, 20 to 70% of Ni, 2 to 4% of Al, 0.005 to 0.4% of rare-earth elements, and at least one member selected from the group consisting of 0.5 to 10% of W and 0.1 to 5% of Mo, the balance being Fe and inevitable impurities; and
a barrier layer formed on a surface of the cast body to be brought into contact with the heated feed-steam mixture;
wherein preferably the barrier layer comprises an Al₂O₃ layer having a thickness of 0.5 μm or more;
wherein preferably at least 80 area % of an outermost surface of the cast body is Al₂O₃;
wherein preferably the cast body has Cr-based particles dispersed at an interface between the Al₂O₃ layer and the cast body at a higher Cr concentration than a Cr concentration of a matrix of the alloy; and
wherein preferably the surface of the cast body before forming Al₂O₃ layer is of a roughness (Ra) of 0.05 to 2.5.

D13. The process of any of D1 to D12, wherein the heated feed-steam mixture of step (C) is supplied to the radiant tube in step (D) through a cross-over pipe, and the heated feed-steam mixture in the cross-over pipe has a temperature in a range from 691° C. to 777° C.

D14. The process of any of D1 to D13, and hydrocarbon cracking occurs in the cross-over pipe.

D15. The process of any of D1 to D14, wherein the ethane-containing hydrocarbon feed comprises ethane at a concentration of at least 50 mol %, based on the total moles of hydrocarbons therein.

D16. The process of any of D1 to D16, wherein:
in step (I), three sequential stages of compression are used, consisting of a first stage, a second stage downstream of the first stage, and a third stage downstream of the second stage;
the first stage has a first stage outlet pressure in a range from 310 kPa-gauge to 960 kPa-gauge;
the second stage has a second stage outlet pressure in a range from 510 kPa-gauge to 1830 kPa-gauge that is higher than the first stage outlet pressure; and
the third stage has a third stage outlet pressure in a range from 930 kPa-gauge to 4200 kPa-gauge that is higher than the second stage outlet pressure.

E1. A process for producing a C2-C4 olefin product from an ethane-containing hydrocarbon feed, the process comprising:
(i) providing the ethane-containing hydrocarbon feed;
(ii) mixing the ethane-containing hydrocarbon feed with a dilution steam to produce a feed-steam mixture;
(iii) heating the feed-steam mixture in a convection section of a steam cracking furnace to obtain a heated feed-steam mixture;
(iv) cracking the heated feed-steam mixture in a radiant tube located in a radiant section of the steam cracking furnace under pyrolysis conditions to product a radiant effluent exiting the cracking furnace having a coil-outlet pressure (“COP”) of from 200 kPa-gauge to 700 kPa-gauge;
(v) cooling the radiant effluent to obtain a cooled effluent;
(vi) feeding at least a portion of the cooled effluent into a quench tower by:
(vi.1) providing a separation device comprising a feed stream inlet, a vapor outlet, and a coke particle deposition section;
(vi.2) feeding the cooled effluent into the feed stream inlet;
(vi.3) obtaining a coke-depleted vapor stream from the vapor outlet as the coke-abated cooled effluent stream; and
(vi.4) obtaining deposited coke from the coke deposition section;
(vii) feeding a quench water stream into the quench tower to contact the cooled effluent in the quench tower;
(viii) obtaining a quench tower overhead vapor stream and a quench tower lower liquid stream from the quench tower;
(ix) processing the quench tower overhead vapor stream to obtain the C2-C4 olefin product by using no more than 3 stages of compression;
(x) separating the first quench tower liquid effluent stream optionally mixed with a tar solvent, to obtain a coke-rich stream, a first aqueous stream, a second aqueous stream, and an oil stream;
(xi) separating from the oil stream a C6-C7 aromatics-rich stream; and
(xii) mixing at least a portion of the C6-C7 aromatics-rich stream with at least one of the following: (a) the first quench tower liquid effluent stream as at least a portion of the tar solvent in step (x); (b) the coke-rich stream after step (x); (c) the aqueous stream after step (x);
(xiii) stripping the second aqueous stream in a stripping column at a column bottom temperature of no greater than 280° F. and a stripping column overhead pressure of no greater than 250 kPa-gauge to obtain a stripping column overhead vapor stream and a stripping column bottoms process water stream;
(xiv) forming a stripping column recycle stream having a higher pressure than the stripping column overhead vapor stream from the stripping column overhead vapor stream; and
(xv) feeding the stripping column recycle stream into the quench tower.

E2. The process of E1, further comprising:
(xvi) removing at least a portion of the coke contained in the first aqueous stream to obtain a coke-depleted water stream; and
(xvii) supplying the coke-depleted water stream as at least a portion of the quench water stream in step (vii).

E3. The process of E1 or E2, wherein step (xiv) comprises at least one of the following:
(xiv.1) feeding the stripping column overhead vapor stream and an ejector motive stream into an ejector to obtain the stripping column recycle stream exiting the ejector; and
(xiv.2) compressing the stripping column overhead vapor stream to obtain the stripping column recycle stream.

E4. The process of any of E1 to E3, further comprising:
(xviii) feeding at least a portion of the stripping column bottoms process water stream into a dilution steam generator; and
(xix) generating at least a portion of the dilution stream of step (ii) from the dilution steam generator.

E5. The process of any of E1 to E4, further comprising:
(xx) removing at least a portion of the coke contained in the first aqueous stream to obtain a coke-depleted water stream; and
(xxi) supplying the coke-depleted water stream as at least a portion of the quench water stream in step (vii).

E6. The process of any of E1 to E5, wherein in step (viii), the first quench tower liquid effluent stream is obtained from the bottom of the quench tower.

E7. The process of any of E1 to E6, wherein in step (viii), the first quench tower liquid effluent stream is obtained from a location above the bottom of the quench tower, and a second quench tower liquid effluent stream is obtained from the bottom of the quench tower.

E8. The process of any of E1 to E7, further comprising at least one of the following: (xxii) mixing the second quench tower liquid effluent stream with a tar solvent.

E9. The process of any of E1 to E8, wherein step (xiii) is carried out at a stripping column overhead pressure from 27 kPa-gauge to 96 kPa-gauge.

E10. The process of any of E1 to E9, wherein in step (iv), the radiant tube comprises:
a cast body of a heat-resistant alloy consisting essentially of, in mass percent, 0.05 to 0.7% of C, more than 0% and no more than 2.5% of Si, more than 0% and no more than 3.0% of Mn, 15 to 50% of Cr, 20 to 70% of Ni, 2 to 4% of Al, 0.005 to 0.4% of rare-earth elements, and at least one member selected from the group consisting of 0.5 to 10% of W and 0.1 to 5% of Mo, the balance being Fe and inevitable impurities; and
a barrier layer formed on a surface of the cast body to be brought into contact with the heated feed-steam mixture;
wherein preferably the barrier layer comprises an Al₂O₃ layer having a thickness of 0.5 μm or more;
wherein preferably at least 80 area % of an outermost surface of the cast body is Al₂O₃;
wherein preferably the cast body has Cr-based particles dispersed at an interface between the Al₂O₃ layer and the cast body at a higher Cr concentration than a Cr concentration of a matrix of the alloy; and
wherein preferably the surface of the cast body before forming Al₂O₃ layer is of a roughness (Ra) of 0.05 to 2.5.

E11. The process of any of E1 to E10, wherein the heated feed-steam mixture of step (iii) is supplied to the radiant tube in step (iv) through a cross-over pipe, and the heated feed-steam mixture in the cross-over pipe has a temperature in a range from 691° C. to 777° C.

E12. The process of any of E1 to E11, and hydrocarbon cracking occurs in the cross-over pipe.

E13. The process of any of E1 to E12, wherein the ethane-containing hydrocarbon feed comprises ethane at a concentration of at least 50 mol %, based on the total moles of hydrocarbons therein.

E14. The process of any of E1 to E13, wherein:
in step (ix), three sequential stages of compression are used, consisting of a first stage, a second stage downstream of the first stage, and a third stage downstream of the second stage;
the first stage has a first stage outlet pressure in a range from 310 kPa-gauge to 960 kPa-gauge;
the second stage has a second stage outlet pressure in a range from 510 kPa-gauge to 1830 kPa-gauge that is higher than the first stage outlet pressure; and
the third stage has a third stage outlet pressure in a range from 930 kPa-gauge to 4200 kPa-gauge that is higher than the second stage outlet pressure.

What is claimed is:

1. A process for producing a C2-C4 olefin product from an ethane-containing hydrocarbon feed, the process comprising:
(I) providing the ethane-containing hydrocarbon feed;
(II) mixing the ethane-containing hydrocarbon feed with a dilution steam to produce a feed-steam mixture;
(III) heating the feed-steam mixture in a convection section of a steam cracking furnace to obtain a heated feed-steam mixture;
(IV) cracking the heated feed-steam mixture in a radiant tube located in a radiant section of the steam cracking furnace under pyrolysis conditions to produce a radiant effluent exiting the steam cracking furnace having a coil-outlet pressure (“COP”) of from 200 kPa-gauge to 700 kPa-gauge;
(V) cooling the radiant effluent to obtain a cooled effluent;
(VI) feeding at least a portion of the cooled effluent into a quench tower;
(VII) feeding a quench water stream into the quench tower to contact the cooled effluent in the quench tower;
(VIII) obtaining a quench tower overhead vapor stream at a location in the vicinity of the top of the quench tower, and a first quench tower liquid effluent stream at a location in the vicinity of the bottom of the quench tower, from the quench tower;
(IX) recovering the C2-C4 olefin product from the quench tower overhead vapor stream by using no more than 3 stages of compression;
(X) separating the first quench tower liquid effluent stream optionally mixed with a tar solvent, to obtain a coke-rich stream, a first aqueous stream, an optional second aqueous stream, and an oil stream;
(XI) removing at least a portion of the coke contained in the first aqueous stream to obtain a coke-depleted water stream; and
(XII) supplying the coke-depleted water stream as at least a portion of the quench water stream in step (VII).

2. The process of claim 1, wherein in step (VIII), the first quench tower liquid effluent stream is obtained from the bottom of the quench tower.

3. The process of claim 1, wherein in step (VIII), the first quench tower liquid effluent stream is obtained from a location above the bottom of the quench tower, and a second quench tower liquid effluent stream is obtained from the bottom of the quench tower.

4. The process of claim 1, further comprising at least one of the following:
(XIII) mixing the coke-rich stream with a tar solvent;
(XIV) mixing the first aqueous stream with a tar solvent;
(XV) mixing the second aqueous stream with a tar solvent; and
(XVI) mixing the second quench tower liquid effluent stream with a tar solvent.

5. The process of claim 1, wherein step (VI) comprises:
(VIa) removing at least a portion of the coke contained in the cooled effluent to obtain a coke-abated cooled effluent stream; and
(VIb) feeding the coke-abated cooled effluent stream as the portion of the cooled effluent into the quench tower.

6. The process of claim 5, wherein step (VIa) comprises:
(VIa.1) providing a separation device comprising a feed stream inlet, a vapor outlet, and a coke particle deposition section;
(VIa.2) feeding the cooled effluent into the feed stream inlet;
(VIa.3) obtaining a coke-depleted vapor stream from the vapor outlet as the coke-abated cooled effluent stream; and
(VIa.4) obtaining deposited coke from the coke deposition section.

7. The process of claim 1, wherein in step (X), the second aqueous stream is obtained, and the process further comprises:
(XIX) stripping the second aqueous stream in a stripping column at a column bottom temperature of no greater than 280° F. and a stripping column overhead pressure of no greater than 250 kPa-gauge to obtain a stripping column overhead vapor stream and a stripping column bottoms process water stream; and
(XX) forming a stripping column recycle stream having a higher pressure than the stripping column overhead vapor stream from the stripping column overhead vapor stream; and
(XXI) feeding the stripping column recycle stream into the quench tower.

8. The process of claim 7, wherein step (XIX) is carried out at a stripping column overhead pressure from 27 kPa-gauge to 96 kPa-gauge.

9. The process of claim 7, further comprising:
(XXII) feeding at least a portion of the stripping column bottoms process water stream into a steam generator; and
(XXIII) generating steam from the dilution steam generator.

10. The process of claim 1, further comprising:
(XVII) separating from at least the oil stream a C6-C7 aromatics-rich stream; and
(XVIII) supplying at least a portion of the C6-C7 aromatics-rich stream as at least a portion of the tar solvent in at least one of steps (X), (XIII), (XIV), (XV), and (XVI).

11. The process of claim 9, wherein at least a portion of the steam generated in step (XXIII) is used as at least portion of the dilution steam of step (II).

12. The process of claim 7, wherein step (XX) comprises at least one of the following:
(XXa) feeding the stripping column overhead vapor stream and an ejector motive stream into an ejector to obtain the stripping column recycle stream exiting the ejector; and
(XXb) compressing the stripping column overhead vapor stream to obtain the stripping column recycle stream.

13. The process of claim 1, wherein in step (IV), the radiant tube comprises:
a cast body of a heat-resistant alloy consisting essentially of, in mass percent, 0.05 to 0.7% of C, more than 0% and no more than 2.5% of Si, more than 0% and no more than 3.0% of Mn, 15 to 50% of Cr, 20 to 70% of Ni, 2 to 4% of Al, 0.005 to 0.4% of rare-earth elements, and at least one member selected from the group consisting of 0.5 to 10% of W and 0.1 to 5% of Mo, the balance being Fe and inevitable impurities; and
a barrier layer formed on a surface of the cast body to be brought into contact with the heated feed-steam mixture.

14. The process of claim 1, wherein the heated feed-steam mixture of step (III) is supplied to the radiant tube in step (IV) through a cross-over pipe, and the heated feed-steam mixture in the cross-over pipe has a temperature in a range from 691° C. to 777° C.

15. The process of claim 1, and hydrocarbon cracking occurs in the cross-over pipe.

16. The process of claim 1, wherein the ethane-containing hydrocarbon feed comprises ethane at a concentration of at least 50 mol %, based on the total moles of hydrocarbons therein.

17. The process of claim 1, wherein:
in step (IX), three sequential stages of compression are used, consisting of a first stage, a second stage downstream of the first stage, and a third stage downstream of the second stage;
the first stage has a first stage outlet pressure in a range from 310 kPa-gauge to 960 kPa-gauge;
the second stage has a second stage outlet pressure in a range from 510 kPa-gauge to 1830 kPa-gauge that is higher than the first stage outlet pressure; and
the third stage has a third stage outlet pressure in a range from 930 kPa-gauge to 4200 kPa-gauge that is higher than the second stage outlet pressure.

18. A process for producing a C2-C4 olefin product from an ethane-containing hydrocarbon feed, the process comprising:
(1) providing the ethane-containing hydrocarbon feed;
(2) mixing the ethane-containing hydrocarbon feed with a dilution steam to produce a feed-steam mixture;
(3) heating the feed-steam mixture in a convection section of a steam cracking furnace to obtain a heated feed-steam mixture;
(4) cracking the heated feed-steam mixture in a radiant tube located in a radiant section of the steam cracking furnace under pyrolysis conditions to produce a radiant effluent exiting the cracking furnace having a coil-outlet pressure (“COP”) of from 200 kPa-gauge to 700 kPa-gauge;
(5) cooling the radiant effluent to obtain a cooled effluent;
(6) feeding at least a portion of the cooled effluent into a quench tower;
(7) feeding a quench water stream into the quench tower to contact the cooled effluent in the quench tower;
(8) obtaining a quench tower overhead vapor stream at a location in the vicinity of the top of the quench tower, and a first quench tower liquid effluent stream at a location in the vicinity of the bottom of the quench tower, from the quench tower;
(9) processing the quench tower overhead vapor stream to obtain the C2-C4 olefin product by using no more than 3 stages of compression;
(10) separating the first quench tower liquid effluent stream optionally mixed with a tar solvent, to obtain a coke-rich stream, a first aqueous stream, a second aqueous stream, and an oil stream; and
(11) stripping the second aqueous stream in a stripping column at a column bottom temperature of no greater than 280° F. and a stripping column overhead pressure of no greater than 250 kPa-gauge to obtain a stripping column overhead vapor stream and a stripping column bottoms process water stream; and
(12) forming a stripping column recycle stream having a higher pressure than the stripping column overhead vapor stream from the stripping column overhead vapor stream; and
(13) feeding the stripping column recycle stream into the quench tower.

19. A process for producing a C2-C4 olefin product from an ethane-containing hydrocarbon feed, the process comprising:
(a) providing the ethane-containing hydrocarbon feed;
(b) mixing the ethane-containing hydrocarbon feed with a dilution steam to produce a feed-steam mixture;
(c) heating the feed-steam mixture in a convection section of a steam cracking furnace to obtain a heated feed-steam mixture;
(d) cracking the heated feed-steam mixture in a radiant tube located in a radiant section of the steam cracking furnace under pyrolysis conditions to product a radiant effluent exiting the cracking furnace having a coil-outlet pressure (“COP”) of from 200 kPa-gauge to 700 kPa-gauge;
(e) cooling the radiant effluent to obtain a cooled effluent;
(f) feeding at least a portion of the cooled effluent into a quench tower by:
(f.1) providing a separation device comprising a feed stream inlet, a vapor outlet, and a coke particle deposition section;
(f.2) feeding the cooled effluent into the feed stream inlet;
(f.3) obtaining a coke-depleted vapor stream from the vapor outlet as the coke-abated cooled effluent stream; and
(f.4) obtaining deposited coke from the coke deposition section;
(g) feeding a quench water stream into the quench tower to contact the cooled effluent in the quench tower;
(h) obtaining a quench tower overhead vapor stream at a location in the vicinity of the top of the quench tower, and a first quench tower liquid effluent stream at a location in the vicinity of the bottom of the quench tower, from the quench tower;
(i) processing the quench tower overhead vapor stream to obtain the C2-C4 olefin product by using no more than 3 stages of compression; and
(j) separating the first quench tower liquid effluent stream optionally mixed with a tar solvent, to obtain a coke-rich stream, a first aqueous stream, a second aqueous stream, and an oil stream.

20. A process for producing a C2-C4 olefin product from an ethane-containing hydrocarbon feed, the process comprising:
(A) providing the ethane-containing hydrocarbon feed;
(B) mixing the ethane-containing hydrocarbon feed with a dilution steam to produce a feed-steam mixture;
(C) heating the feed-steam mixture in a convection section of a steam cracking furnace to obtain a heated feed-steam mixture;
(D) cracking the heated feed-steam mixture in a radiant tube located in a radiant section of the steam cracking furnace under pyrolysis conditions to product a radiant effluent exiting the cracking furnace having a coil-outlet pressure (“COP”) of from 200 kPa-gauge to 700 kPa-gauge;
(E) cooling the radiant effluent to obtain a cooled effluent;
(F) feeding at least a portion of the cooled effluent into a quench tower;
(G) feeding a quench water stream into the quench tower to contact the cooled effluent in the quench tower;
(H) obtaining a quench tower overhead vapor stream at a location in the vicinity of the top of the quench tower, and a first quench tower liquid effluent stream at a location in the vicinity of the bottom of the quench tower, from the quench tower;
(I) processing the quench tower overhead vapor stream to obtain the C2-C4 olefin product by using no more than 3 stages of compression;
(J) separating the first quench tower liquid effluent stream optionally mixed with a tar solvent, to obtain a coke-rich stream, a first aqueous stream, a second aqueous stream, and an oil stream; and
(K) separating from the oil stream a C6-C7 aromatics-rich stream; and
(L) mixing at least a portion of the C6-C7 aromatics-rich stream with at least one of the following: (i) the first quench tower liquid effluent stream as at least a portion of the tar solvent in step (J); (ii) the coke-rich stream after step (J); (iii) the first and/or second aqueous streams after step (J).

21. A process for producing a C2-C4 olefin product from an ethane-containing hydrocarbon feed, the process comprising:
(i) providing the ethane-containing hydrocarbon feed;
(ii) mixing the ethane-containing hydrocarbon feed with a dilution steam to produce a feed-steam mixture;
(iii) heating the feed-steam mixture in a convection section of a steam cracking furnace to obtain a heated feed-steam mixture;
(iv) cracking the heated feed-steam mixture in a radiant tube located in a radiant section of the steam cracking furnace under pyrolysis conditions to product a radiant effluent exiting the cracking furnace having a coil-outlet pressure (“COP”) of from 200 kPa-gauge to 700 kPa-gauge;
(v) cooling the radiant effluent to obtain a cooled effluent;
(vi) feeding at least a portion of the cooled effluent into a quench tower by:
(vi.1) providing a separation device comprising a feed stream inlet, a vapor outlet, and a coke particle deposition section;
(vi.2) feeding the cooled effluent into the feed stream inlet;
(vi.3) obtaining a coke-depleted vapor stream from the vapor outlet as the coke-abated cooled effluent stream; and
(vi.4) obtaining deposited coke from the coke deposition section;
(vii) feeding a quench water stream into the quench tower to contact the cooled effluent in the quench tower;
(viii) obtaining a quench tower overhead vapor stream and a quench tower lower liquid stream from the quench tower;
(ix) processing the quench tower overhead vapor stream to obtain the C2-C4 olefin product by using no more than 3 stages of compression;
(x) separating the first quench tower liquid effluent stream optionally mixed with a tar solvent, to obtain a coke-rich stream, a first aqueous stream, a second aqueous stream, and an oil stream;
(xi) separating from the oil stream a C6-C7 aromatics-rich stream; and
(xii) mixing at least a portion of the C6-C7 aromatics-rich stream with at least one of the following: (a) the first quench tower liquid effluent stream as at least a portion of the tar solvent in step (x); (b) the coke-rich stream after step (x); (c) the first and second aqueous stream after step (x);
(xiii) stripping the second aqueous stream in a stripping column at a column bottom temperature of no greater than 280° F. and a stripping column overhead pressure of no greater than 250 kPa-gauge to obtain a stripping column overhead vapor stream and a stripping column bottoms process water stream;
(xiv) forming a stripping column recycle stream having a higher pressure than the stripping column overhead vapor stream from the stripping column overhead vapor stream; and
(xv) feeding the stripping column recycle stream into the quench tower.

22. The process of claim 21, further comprising:
(xvi) removing at least a portion of the coke contained in the first aqueous stream to obtain a coke-depleted water stream; and
(xvii) supplying the coke-depleted water stream as at least a portion of the quench water stream in step (vii).

23. The process of claim 21, wherein step (xiv) comprises at least one of:
(xiv.1) feeding the stripping column overhead vapor stream and an ejector motive stream into an ejector to obtain the stripping column recycle stream exiting the ejector; and
(xiv.2) compressing the stripping column overhead vapor stream to obtain the stripping column recycle stream.

24. The process of claim 21, further comprising:
(xviii) feeding at least a portion of the stripping column bottoms process water stream into a dilution steam generator; and
(xix) generating at least a portion of the dilution stream of step (ii) from the dilution steam generator.

25. The process of claim 21, wherein in step (viii), the first quench tower liquid effluent stream is obtained from the bottom of the quench tower.

26. The process of claim 21, wherein in step (viii), the first quench tower liquid effluent stream is obtained from a location above the bottom of the quench tower, and a second quench tower liquid effluent stream is obtained from the bottom of the quench tower.

27. The process of claim 21, further comprising at least one of the following:
(xxii) mixing the second quench tower liquid effluent stream with a tar solvent.

28. The process of claim 21, wherein step (xiii) is carried out at a stripping column overhead pressure from 27 kPa-gauge to 96 kPa-gauge.

29. The process of claim 21, wherein in step (iv), the radiant tube comprises:
a cast body of a heat-resistant alloy consisting essentially of, in mass percent, 0.05 to

0. 7% of C, more than 0% and no more than 2.5% of Si, more than 0% and no more than 3.0% of Mn, 15 to 50% of Cr, 20 to 70% of Ni, 2 to 4% of Al, 0.005 to 0.4% of rare-earth elements, and at least one member selected from the group consisting of 0.5 to 10% of W and 0.1 to 5% of Mo, the balance being Fe and inevitable impurities; and
a barrier layer formed on a surface of the cast body to be brought into contact with the heated feed-steam mixture.

30. The process of claim 21, wherein the heated feed-steam mixture of step (iii) is supplied to the radiant tube in step (iv) through a cross-over pipe, and the heated feed-steam mixture in the cross-over pipe has a temperature in a range from 691° C. to 777° C.

31. The process of claim 21, and hydrocarbon cracking occurs in the cross-over pipe.

32. The process of claim 21, wherein the ethane-containing hydrocarbon feed comprises ethane at a concentration of at least 50 mol %, based on the total moles of hydrocarbons therein.

33. The process of claim 21, wherein:
in step (ix), three sequential stages of compression are used, consisting of a first stage, a second stage downstream of the first stage, and a third stage downstream of the second stage;
the first stage has a first stage outlet pressure in a range from 310 kPa-gauge to 960 kPa-gauge;
the second stage has a second stage outlet pressure in a range from 510 kPa-gauge to 1830 kPa-gauge that is higher than the first stage outlet pressure; and
the third stage has a third stage outlet pressure in a range from 930 kPa-gauge to 4200 kPa-gauge that is higher than the second stage outlet pressure.
